SEQUENCE LISTING (2 sequences)

SEQ ID NO: 1 - protein, 400 aa, Homo Sapien [sequence omitted]

SEQ ID NO: 2 - DNA, 1203 bp, Homo Sapien
atggattcacttggcgccgtcagcactcgacttgggtttgatcttttcaaagagctgaagaaaacaaatgatggcaacatcttcttttcccctgtgggcatcttgactgcaattggcatggtcctcctggggacccgaggagccaccgcttcccagttggaggaggtgtttcactctgaaaaagagacgaagagctcaagaataaaggctgaagaaaaagaggtggtaagaataaaggctgaaggaaaagagattgagaacacagaagcagtacatcaacaattccaaaagtttttgactgaaataagcaaactcactaatgattatgaactgaacataaccaacaggctgtttggagaaaaaacatacctcttccttcaaaaatacttagattatgttgaaaaatattatcatgcatctctggaacctgttgattttgtaaatgcagccgatgaaagtcgaaagaagattaattcctgggttgaaagcaaaacaaatgaaaaaatcaaggacttgttcccagatggctctattagtagctctaccaagctggtgctggtgaacatggtttattttaaagggcaatgggacagggagtttaagaaagaaaatactaaggaagagaaattttggatgaataagagcacaagtaaatctgtacagatgatgacacagagccattcctttagcttcactttcctggaggacttgcaggccaaaattctagggattccatataaaaacaacgacctaagcatgtttgtgcttctgcccaacgacatcgatggcctggagaagataatagataaaataagtcctgagaaattggtagagtggactagtccagggcatatggaagaaagaaaggtgaatctgcacttgccccggtttgaggtggaggacagttacgatctagaggcggtcctggctgccatggggatgggcgatgccttcagtgagcacaaagccgactactcgggaatgtcgtcaggctccgggttgtacgcccagaagttcctgcacagttcctttgtggcagtaactgaggaaggcaccgaggctgcagctgccactggcataggctttactgtcacatccgccccaggtcatgaaaatgttcactgcaatcatcccttcctgttcttcatcaggcacaatgaatccaacagcatcctcttcttcggcagattttcttctccttaa

(19) United States
(12) Patent Application Publication
(10) Pub. No.: US 2015/0306193 A1
(43) Pub. Date: Oct. 29, 2015

(54) COMPOSITIONS AND METHODS FOR TREATING IMMUNE CONDITIONS, INCLUDING TYPE 1 DIABETES

(71) Applicant: Jan CZYZYK, Rochester, NY (US)

(72) Inventor: Jan CZYZYK, Rochester, NY (US)

(21) Appl. No.: 14/648,597

(22) PCT Filed: Nov. 29, 2013
(86) PCT No.: PCT/US2013/072491

§ 371 Date: May 29, 2015

Related U.S. Application Data
(60) Provisional application No. 61/731,444, filed on Nov. 29, 2012.

(51) Int. Cl. A61K 39/00; A61K 39/385; C07K 14/77; A61K 39/39

(52) U.S. Cl. CPC A61K 39/0005; A61K 39/39; A61K 39/385; C07K 14/77; A61K 2039/55505; C07K 2319/00; A61K 2039/55516

(57) ABSTRACT

The present invention provides a vaccine, immunogenic polypeptide, and pharmaceutical compound, all including a serpinB13 protein or polypeptide or an immunogenic polypeptide that includes a serpinB13 protein or polypeptide coupled to an immunogenic agent. The present invention further includes use of these agents according to a method of inhibiting or delaying onset, or reducing the severity of type 1 diabetes, as well as a method of treating an individual for an immune condition selected from the group of psoriasis, hair loss, and ulcers including diabetic food ulceration.

This application claims benefit of U.S. Provisional Patent Application Ser. No. 61/731,444, filed on Nov. 29, 2012, which is hereby incorporated by reference in its entirety.

### FIELD OF THE INVENTION

The present invention relates generally to compositions and methods for treating immune conditions including Type 1 Diabetes.

### BACKGROUND OF THE INVENTION

One-third of diabetes patients suffer from Type 1 Diabetes (“T1D”) (Foster et al., Harrison's Principles of Internal Medicine, Chap. 114, pp. 661-678, 10th Ed., McGraw-Hill, New York). T1D is an autoimmune disease wherein a state of hyperglycemia results from the T-cell mediated destruction of insulin-secreting beta cells in the pancreatic Islets of Langerhans (Eisenbarth et al., Type I Diabetes Mellitus. A Chronic Autoimmune Disease,” New Engl. J. Med. 314:1360-1368 (1986)). The disease manifests itself as a series of hormone-induced metabolic abnormalities which eventually lead to serious, long-term, and debilitating complications involving several organ systems including the eyes, kidneys, nerves, and blood vessels. Pathologically, the disease is characterized by lesions of the basement membranes, demonstrable under electron microscopy.

Individuals with T1D characteristically show very low or immeasurable plasma insulin with elevated glucagon. Regardless of what the exact etiology is, most T1D patients have circulating antibodies directed against their own pancreatic cells including antibodies to insulin, to the islet of Langerhans cell cytoplasm and to the enzyme glutamic acid decarboxylase. An immune response specifically directed against beta cells (insulin producing cells) leads to T1D.

Traditional approaches to T1D therapy are based on attempts to inhibit autoimmune inflammation in the pancreatic islets, or alternatively, to improve beta cell renewal. These approaches are dependent on targeting distinct molecular pathways, and consequently, require the use of reagents with equally distinct target specificities. Other therapeutic regimens for T1D include modifications to the diet to minimize hyperglycemia resulting from the lack of natural insulin, which in turn, is the result of damaged beta cells. Diet is also modified with regard to insulin administration to counter the hypoglycemic effects of the hormone.

Despite many years of research, a cure for T1D has not been found. Today the disease is primarily managed by receiving exogenous insulin through injections or an insulin pump. Therefore, there is an unmet need for novel therapies that focus on beta cells and reversal of autoimmunity. According to recent recommendation by experts in the field of autoimmune diabetes, combination therapy that suppresses T-cell-mediated autoimmunity and nonspecific inflammation while enhancing beta cell survival to control T1D holds more promise than conventional monotherapies (Matthews et al., “Developing Combination Immunotherapies for Type 1 Diabetes: Recommendations From the ITN-JDRF Type 1 Diabetes Combination Therapy Assessment Group,” Clin. Exp. Immunol. 160:176-184 (2010)).

The present invention is directed to overcoming these deficiencies in the art by enhances anti-serpin immunological response.

### SUMMARY OF THE INVENTION

A first aspect of the present invention relates to a vaccine that includes a pharmaceutically acceptable carrier; and a serpinB13 protein or polypeptide.

A second aspect of the present invention relates to an immunogenic polypeptide that includes a serpinB13 protein or polypeptide coupled to an immunogenic agent.

A third aspect of the present invention relates to a pharmaceutical composition that includes the immunogenic polypeptide according to the second aspect of the invention.

A fourth aspect of the present invention relates to a method of inhibiting or delaying onset, or reducing the severity of, type 1 diabetes. This method includes administering to an individual having a risk of developing type 1 diabetes an effective amount of:
(i) serpinB13 protein or a polypeptide fragment thereof,
(ii) a vaccine according to the first aspect of the invention,
(iii) an immunogenic polypeptide according to the second aspect of the invention, or
(iv) a pharmaceutical composition according to the third aspect of the invention, wherein the administering is effective to induce an anti-serpinB13 antibody response that inhibits or delays onset of type 1 diabetes, or reduces the severity of type 1 diabetes in the individual.

A fifth aspect of the present invention relates to a method of treating an individual for an immune condition that includes administering to an individual having an immune condition an effective amount of:
(i) serpinB13 protein or a polypeptide fragment thereof,
(ii) a vaccine according to the first aspect of the invention,
(iii) an immunogenic polypeptide according to the second aspect of the invention, or
(iv) a pharmaceutical composition according to the third aspect of the invention, wherein the administering is effective to treat the immune condition or control symptoms thereof, and wherein the immune condition is selected from the group consisting of psoriasis, hair loss, and ulcers including diabetic food ulceration.

The present invention identifies an immune response against the protease inhibitor serpinB13 that both suppresses T-cell mediated autoimmune inflammation and enhances beta cell survival and therefore represents a novel therapeutic or inhibitory intervention for T1D (Czyzyk et al., “Enhanced Anti-Serpin Antibody Activity Inhibits Autoimmune Inflammation in Type 1 Diabetes,” J. Immunol. 188:6319-6327 (2012); Baldzizhar et al., “Anti-Serpin Antibody-Mediated Regulation of Proteases in Autoimmune Diabetes,” J. Biol. Chem. 288:1612-1619 (2013), both of which are hereby incorporated by reference in their entirety). These findings are relevant to T1D in humans because analysis of human samples demonstrated that early-onset (age<5 years) T1D is associated with a low output of anti-serpin B13 autoantibodies suggesting, an inverse relationship between the anti-serpin B13 immunological response and the development of clinical diabetes. It is proposed that stimulating anti-serpin immunity may delay or even reverse T1D. Thus, in the long term, development of experimental protocols that employ passive or active immunization against serpins may prompt development of anti-serpin vaccine to control diabetes that is safe, therapeutically beneficial, and cost-effective.

The present invention also targets serpinB13 therapeutically to promote islet regeneration and inhibit the inflammatory response in T1D. It was recently discovered that a novel autoantibody (AA) against the protease inhibitor serpinB13 (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety) that is associated with low anti-insulin AAs levels, reduced T-cell infiltration of pancreatic islets, and delayed onset of diabetes in non-obese diabetic (“NOD”) mice. A monoclonal antibody (mAb) against serpinB13 also reduces islet inflammation and accelerates recovery from diabetes in NOD mice while enhancing the expression of genes associated with islet endocrine cell differentiation in Balb/c mice with streptozotocin (STZ)-induced diabetes. Because the natural anti-serpin immune response is prominent early in life, it is believed that progression of diabetes can be modified by stimulating anti-serpin immunity in young animals by injecting them with serpinB13. Support for this hypothesis is provided by reduced glycemia following serpinB13 administration in young Balb/c with STZ-induced diabetes.

The incidence of T1D is increasing faster than before (Patterson et al., “EURODIAB Study Group, Incidence Trends for Childhood Type 1 Diabetes in Europe During 1989-2003 and Predicted New Cases 2005-2020: A Multicentre Prospective Registration Study,” Lancet 373:2027-2033 (2009); Harjutsalo et al., “Time Trends in the Incidence of Type 1 Diabetes in Finnish Children: A Cohort Study,” Lancet 371:1777-1782 (2008), both of which are hereby incorporated by reference in their entirety), especially in very young children lacking an anti-serpin immune response. Enhanced serpin immunity may help reverse this trend by promoting islet renewal and inhibiting islet inflammation. This approach is consistent with the recommendation of using combination therapy that suppresses T-cell-mediated autoimmunity and nonspecific inflammation while enhancing beta cell survival to control T1D (Matthews et al., “Developing Combination Immunotherapies for Type 1 Diabetes: Recommendations From the ITN-JDRF Type 1 Diabetes Combination Therapy Assessment Group,” Clin. Exp. Immunol. 160:176-184 (2010), which is hereby incorporated by reference in its entirety).

### BRIEF DESCRIPTION OF THE DRAWINGS

FIGS. 1A-D show expression of serpinB13 in the pancreas. Costaining of pancreatic frozen sections obtained from 6-week-old NOD mouse with anti-serpinB13 mAb and antibodies directed against glucagon are shown in FIG. 1A, for CD31 in FIG. 1B, and for keratin 19 in FIG. 1C. Staining with the isotype control IgG2b failed to produce the pattern seen with anti-serpinB13 mAb (FIG. 1A, left). FIG. 1D shows time course of serpinB13 expression at a young age. Pancreata from 4-, 14-, and 21-day-old NOD mice were stained with mAbs against serpinB13 (1D upper) and keratin 19 (1D lower). Scale bar, 50 μm (FIGS. 1A and 1D) and 100 pm (FIGS. 1B and 1C).

FIGS. 2A-C illustrate the effect of anti-serpinB13 mAb on protease target. In FIG. 2A, serum binding activity in NOD mice to serpinB13 was isolated from different sources. In FIG. 2A, left panel, serum samples that were positive (S1, S2, and S3, n=3) or negative (S4, S5, and S6, n=3) for binding to serpinB13 produced in 293T cells were analyzed to determine the binding characteristics of this molecule produced in insect cells and Escherichia coli. The assay was performed exactly as described previously in Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety), and data are expressed as FI units and represent the total FI minus FI due to serum-binding activity in the presence of beads precoated with a control lysate (293 T cells transfected with GFP). Luminex beads were loaded with 10 to 20 μg of purified protein or 1 to 2 mL of cell lysates. In FIG. 2A, the right panel shows Western blot analysis of purified proteins (0.5 μg of protein per lane in lane 1 and 2) or cell lysates corresponding to 293T cells transfected with serpinB13 of GFP (50 μL of cell lysate per lane in lane 3 and 4) are shown that were used to perform serum binding activity assay depicted in the left panel. The blot was stained with anti-His₍₆₎ polyclonal antibody. In FIG. 2B, the left panel shows the cleavage of (CBZ-Phe-Arg)-R110 substrate by cathepsin L in the presence of serpinB13 and anti-serpinB13 mAb is indicated. Data are expressed as fold induction of maximum fluorescence over background (which was measured in the presence of the substrate alone). The average of 3 experiments described is shown. In FIG. 2B, the right panel shows Western blot analysis of serpinB13 staining with mAb raised against mouse serpinB13 is shown. The lysates of 293T cells transfected with either an empty vector (lane 1) or mouse serpinB13 (lane 2) were analyzed. FIG. 2C shows the in vivo effect of anti-serpinB13 mAb on cathepsin L in the pancreas of female NOD mice treated with anti-serpinB13 mAb (n=4) or control IgG (n=4). Four-week-old animals were injected 4 times i.v. (100 μg/injection) over a 10-day period. Representative analysis is depicted in the left graph, and results from three independent experiments are summarized on the right graph of FIG. 2C. The error bars indicate standard deviation. FU-fluorescence units.

FIGS. 3A-B show the effect of anti-serpinB13 mAb on CD4 and CD19 in the pancreas-associated lymphocytes. FIG. 3A, the left graphs show FACS analysis of CD4 expression in the islets and inguinal lymph nodes of NOD mice treated with IgG control (n=4), anti-serpinB13 mAb (n=4) E64-protease inhibitor (n=4), or both anti-serpinB13 and E64 inhibitor (n=4). Over a 10-day period, 4-week-old mice (prescreened for the low levels of anti-serpin autoantibodies) were injected 4 times i.v. with mAb or control IgG (100 μg/injection) and 10 times i.p. with E64 (10 mg/kg) or diluent, as indicated. Animals were sacrificed and cell suspensions obtained from their organs were stained with PE-conjugated anti-CD4 mAb at 1:800 (this dilution of anti-CD4 mAb allowed us to distinguish between high (M1) and low (M2) rates of expression of CD4 in T cells. In FIG. 3A, the right graphs show the average of 3 experiments. Data are expressed as the ratio between populations with low and high CD4 expression. In FIG. 3B, the left graphs show FACS analysis of B220 and CD19 expression in the pancreatic and inguinal lymph nodes of NOD mice treated with anti-serpinB13 mAb and/or E64 (n=8), exactly as described in FIG. 3A. Animals were sacrificed and their lymph nodes were stained with PE-conjugated anti-B220 (1:200) and FITC-conjugated anti-CD19 mAb (1:100). The M1 and M2 regions depict high and low CD19 expressors, respectively. FIG. 3B on the right shows the average of 3 experiments described. Data are expressed as the ratio between populations with low versus high rates of CD19 expression.

FIG. 4 illustrates the effect of anti-serpinB13 natural autoantibodies on CD4 in the pancreas-associated lymphocytes. Shown in FIG. 4 is an analysis of CD4 expression in 4-week-old female NOD mice that had been prescreened for low (SBA^{low}) or high (SBA^{high}) secretion of anti-serpinB13 autoantibodies and received either E64 or diluent (PBS) for 10 consecutive days, exactly as described in the legend to FIG. 3A. The animals were sacrificed and cell suspensions obtained from their organs were stained with PE-conjugated anti-CD4 mAb at 1:800; this dilution of anti-CD4 mAb allowed us to distinguish between high (M1) and low (M2) rates of expression of CD4 in T cells. Each histogram was generated by examining 4 animals. FIG. 4, on the right shows the average of 3 experiments described. The data are expressed as the ratio between populations with low versus high rates of CD4 expression. The errors bars indicate standard deviation. SBA, anti-serpinB13 autoantibodies.

FIGS. 5A-B depict the cleavage of CD4 and CD19 in the pancreatic lymph nodes. Western blot analysis of CD4 and CD19 in cells from the inguinal and pancreatic lymph nodes are shown, sorted for the high (R1 and R2) and intermediate/low (R3) levels of these markers. The blots were stained with an anti-CD 19 antibody that recognizes the cytoplasmic portion of the molecule (FIG. 5A) or two different anti-CD4 antibodies (FIG. 5B) that recognize the intracellular (C-18) or extracellular (J15) portion of CD4. The control blot (FIG. 5A, right panel) was stained with the secondary reagent only. The data is representative of three independent experiments.

FIGS. 6A-B illustrate diminished secretion of IFN-γ in T cells with cleaved form of CD4 (CD4^{low}) molecule. CD4^{high} and CD4^{low} T cells were isolated from the PLNs of BDC2.5 TCR transgenic NOD mice by sorting of cells that positively stained with FITC-conjugated anti-Vβ4 TCR chain mAb (1:500) and PE-conjugated anti-CD4 mAb (1:800). The cells were then stimulated with different concentrations of BDC2.5 mimotope in the presence of antigen presenting cells (APCs) (FIG. 6A, left graph) or phorbol esters (PMA) and ionomycin, as indicated (FIG. 6A, right graph). At 48 hours after initiation of stimulation, the cells were counted and culture supernatants were examined by ELISA for IFN-γ concentration. The average of 3 independent experiments described is shown. In FIG. 6B, the cells after stimulation for 72 hours with the BDC2.5 mimotope-pulsed APCs were rested for 48 hours and then restimulated with PMA (10 ng/mL) and ionomycin (1 μM). The StopGolgi reagent was added during the last 8 hours of secondary culture. The cytokine production was examined by an intracellular staining of cells with APC-conjugated mAb against IFNγ (1:100). The data are representative of three independent experiments. The error bars indicate standard deviation.

FIG. 7 shows a western blot analysis of purified keratin 19 (lane 1) and serpinB13 (lane 2). A 0.5 μg sample of protein from each lane was analyzed. The blot was stained with anti-serpinB13 mAb (1 μg/mL) followed by horseradish peroxidase (HRP)-linked goat anti-mouse secondary antibody (1:10000).

FIG. 8 depicts expression of lymphocytes surface markers after treatment with cathepsin L. Splenocytes isolated from 6-week-old female NOD mice were treated with cathepsin L (1 μg/mL) in an appropriate bugger (150 mM NaCl, 50 mM sodium acetate, pH 5.5, 4 mM DTT, and 1 mM EDTA [green]) or buffer alone (purple) at 37° C. for 45 min. The cells were then washed with PBS and stained with individual monoclonal antibodies, exactly as indicated. TCR, T-cell receptor; LCA, leukocyte common antigen.

FIG. 9 shows the effect of treatment with anti-serpinB13 mAb on streptozotocin (“STZ”)-induced diabetes in Balb/c mice. 8-day old male mice were injected with STZ (100 mg/kg) and then i.v. treated with 2.5 μg of anti-serpin mAb or IgG control on day 11, 13, 16, and 18. The animals were monitored for blood glucose at 6 weeks of age and every 7 days thereafter.

FIGS. 10A-B show relative gene expression in the islets. Balb/c mice (FIG. 10A) treated with STZ and anti-serpin mAb (red) or control IgG (dots), and NOD mice (FIG. 10B) with (red) or without (dots) endogenous anti-serpin autoantibodies were examined for gene expression by qPCT, in order as indicated. All mice were 5 weeks old at the time of analysis. A-Arx, B-Brn4, c-Hlxb9, d-Insm1, e-Irx1, f-Irx2, g-Isl1, h-MafA, i-Nkx2.2, j-Pax4, k-Pax6, 1-Neurod1, m-Ins2, n-Ngn3, o-nkx6.1, p-Pdx1, q-Reg1, r-Reg2, s-Reg3a, t-Reg3b, u-Reg3d, v-Reg3g, w-Reg4.

FIG. 11 shows the effect of serpin injection on diabetes induced with STZ. Balb/c newborn male mice were injected on two occasions (Day +5 and +10) with 5-to-10 micrograms of mouse serpin B13 (GenScript) diluted in PBS or PBS alone and then injected with a single dose of 100 mg/kg streptozotocin (STZ) on Day +10 to induce diabetes. Mice were monitored for blood glucose levels at 1 week intervals, starting on Day +17, as indicated.

FIGS. 12A-B shows the effect of immunization with serpin B13 on streptozotocin-induced diabetes in Balb/c mice. Secretion of anti-serpin antibodies after immunization of mice with different doses of antigen is indicated in FIG. 12A. Balb/c newborn male mice were injected on Day +5 and +10 with PBS (control) or 0.1, 1.0 or 10 micrograms of purified mouse serpin B13 (GenScript), as indicated. Mice were then screened for the presence of anti-serpin immunoglobulins using Luminex assay (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety). The assay was performed on serum samples collected on Day +21 and Day +46. Blood glucose levels in mice productively immunized with serpin B13 (red) are shown in FIG. 12B. Balb/c male mice were treated with serpin B13 exactly as described in A, and then injected with a single dose of STZ (100 mg/dL) and weekly monitored for blood glucose levels. These animals were also bled on Day +21 and +46 to monitor anti-serpin antibodies as a readout of immunization. Animals immunized but failing to secrete antibodies (orange) demonstrated similar glucose levels to control animals treated with PBS.

FIG. 13 illustrates an improved response to immunization with serpin B13 and alum adjuvant. Balb/c newborn male mice were injected on day +5 and +10 with 100 microliters of alum adjuvant or a combination of alum and 0.1, 1.0, or 10 micrograms of purified mouse serpin B13 (GenScript). The control mice received PBS. Mice were then screened for the presence of anti-serpin immunoglobulins using Luminex assay. The assay was performed on serum samples collected on Day +21 and Day +46.

### DETAILED DESCRIPTION OF THE INVENTION

The therapeutic agents of the present invention, i.e., vaccine, immunogenic conjugate, or pharmaceutical composition, includes or is derived from the structure of serpinB13, which will induce the formation of anti-serpinB13 antibodies in individuals to inhibit or delay onset of type 1 diabetes, or reduce the severity of type 1 diabetes in the individual. In some circumstances, it is possible that type 1 diabetes, and the damage caused by it, can be reversed.

Human SerpinB13 is disclosed at Genbank Accessions BC143402 (nt) and AAI43403 (prt), which are hereby incorporated by reference in their entirety. The serpinB13 protein or polypeptide may be a human serpinB13 protein of polypeptide. Human serpinB13 has the amino acid sequence (SEQ ID NO: 1) shown below:

MDSLGAVSTRLGFDLFKELKKTNDGNIFFSPVGILTAIGMVLLGTRGATA
SQLEEVFHSEKETKSSRIKAEEKEVVRIKAEGKEIENTEAVHQQFQKFLT
EISKLTNDYELNITNRLFGEKTYLFLQKYLDYVEKYYHASLEPVDFVNAA
DESRKKINSWVESKTNEKIKDLFPDGSISSSTKLVLVNMVYFKGQWDREF
KKENTKEEKFWMNKSTSKSVQMMTQSHSFSFTFLEDLQAKILGIPYKNND
LSMFVLLPNDIDGLEKIIDKISPEKLVEWTSPGHMEERKVNLHLPRFEVE
DSYDLEAVLAAMGMGDAFSEHKADYSGMSSGSGLYAQKFLHSSFVAVTEE
GTEAAAATGIGFTVTSAPGHENVHCNHPFLFFIRHNESNSILFFGRFSSP

This protein is encoded by the nucleotide sequence (SEQ ID NO: 2) shown below:

atggattcacttggcgccgtcagcactcgacttgggtttgatcttttcaa
agagctgaagaaaacaaatgatggcaacatcttcttttcccctgtgggca
tcttgactgcaattggcatggtcctcctggggacccgaggagccaccgct
tcccagttggaggaggtgtttcactctgaaaaagagacgaagagctcaag
aataaaggctgaagaaaaagaggtggtaagaataaaggctgaaggaaaag
agattgagaacacagaagcagtacatcaacaattccaaaagtttttgact
gaaataagcaaactcactaatgattatgaactgaacataaccaacaggct
gtttggagaaaaaacatacctcttccttcaaaaatacttagattatgttg
aaaaatattatcatgcatctctggaacctgttgattttgtaaatgcagcc
gatgaaagtcgaaagaagattaattcctgggttgaaagcaaaacaaatga
aaaaatcaaggacttgttcccagatggctctattagtagctctaccaagc
tggtgctggtgaacatggtttattttaaagggcaatgggacagggagttt
aagaaagaaaatactaaggaagagaaattttggatgaataagagcacaag
taaatctgtacagatgatgacacagagccattcctttagcttcactttcc
tggaggacttgcaggccaaaattctagggattccatataaaaacaacgac
ctaagcatgtttgtgcttctgcccaacgacatcgatggcctggagaagat
aatagataaaataagtcctgagaaattggtagagtggactagtccagggc
atatggaagaaagaaaggtgaatctgcacttgccccggtttgaggtggag
gacagttacgatctagaggcggtcctggctgccatggggatgggcgatgc
cttcagtgagcacaaagccgactactcgggaatgtcgtcaggctccgggt
tgtacgcccagaagttcctgcacagttcctttgtggcagtaactgaggaa
ggcaccgaggctgcagctgccactggcataggctttactgtcacatccgc
cccaggtcatgaaaatgttcactgcaatcatcccttcctgttcttcatca
ggcacaatgaatccaacagcatcctcttcttcggcagattttcttctcct
taa

As an alternative to administering serpinB13, polypeptide fragments of serpinB13 can be utilized. The polypeptide fragments preferably contain at least 10 consecutive amino acids, alternatively at least 15, 20, 25, 30, 35, 40, 45, or 50 consecutive amino acids from SEQ ID NO: 1 listed above, and promote the formation of antibodies that bind specifically to serpinB13 and inhibit its activity.

The full length serpinB13 or polypeptide fragments thereof can be presented to the immune system of the recipient in the form of an immunogenic conjugate or an immunogenic fusion protein, as discussed below.

A fusion protein of the invention includes any one of the serpinB13 polypeptide fragments of the present invention linked by an in-frame fusion to an adjuvant polypeptide. The adjuvant polypeptide can be any peptide adjuvant known in art including, but not limited to, flagellin, human papillomavirus (HPV) L1 or L2 proteins, herpes simplex glycoprotein D (gD), complement C4 binding protein, toll-like receptor-4 (TLR4) ligand, and IL-1β.

The fusion proteins of the present invention can be generated using standard techniques known in the art. For example, the fusion polypeptide can be prepared by translation of an in-frame fusion of the polynucleotide sequences encoding the serpinB13 protein or polypeptide fragment (described infra) and the adjuvant, i.e., a hybrid gene. The hybrid gene encoding the fusion polypeptide is inserted into an expression vector which is used to transform or transfect a host cell. Alternatively, the polynucleotide sequence encoding the serpinB13 protein or polypeptide fragment is inserted into an expression vector in which the polynucleotide encoding the adjuvant is already present. The peptide adjuvant of the fusion protein can be fused to the N-, or preferably, to the C-terminal end of the serpinB13 protein or polypeptide fragment. The serpinB13 polypeptide may be a terminal or internal fragment comprising at least about 10 consecutive peptides, alternatively at least about 15, 20, 25, 30, 35, 40, 45, or 50 consecutive peptides.

Fusions between the serpinB13 protein or polypeptide fragment and the protein adjuvant may be such that the amino acid sequence of the serpinB13 protein or polypeptide fragment is directly contiguous with the amino acid sequence of the adjuvant. Alternatively, the serpinB13 protein or polypeptide fragment may be coupled to the adjuvant by way of a short linker sequence. Suitable linker sequences include glycine rich linkers (e.g., GGGS₂-₃), serine-rich linkers (e.g., GS_{N}), or other flexible immunoglobulin linkers as disclosed in U.S. Pat. No. 5,516,637 to Huang et al, which is hereby incorporated by reference in its entirety.

Recombinant DNA molecules encoding the serpinB13 protein or polypeptide, or a fusion protein containing the same can also be administered in the form of a DNA vaccine, which will induce expression of the polypeptide or fusion protein, as desired.

Another aspect of the present invention is directed to an immunogenic conjugate including the serpinB13 protein or polypeptide fragment conjugated to an immunogenic carrier molecule. Suitable immunogenic conjugates of the present invention include, but are not limited to, an immunogenic carrier molecule covalently or non-covalently bonded to any one of the polypeptides of the present invention. Any suitable immunogenic carrier molecule can be used. Exemplary immunogenic carrier molecules include, but are in no way limited to, bovine serum albumin, chicken egg ovalbumin, keyhole limpet hemocyanin, tetanus toxoid, diphtheria toxoid, thyroglobulin, a pneumococcal capsular polysaccharide, CRM 197, and a meningococcal outer membrane protein. The vaccine may include a serpinB13 protein or polypeptide that is conjugated to an immunogenic carrier molecule.

The vaccine includes a pharmaceutically acceptable carrier. The vaccine or pharmaceutical composition may also include excipients or diluents.

Solutions or suspensions of these active agents can be prepared in water suitably mixed with a surfactant, such as hydroxypropylcellulose. Dispersions can also be prepared in glycerol, liquid polyethylene glycols, and mixtures thereof in oils.

Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils. Formulations suitable for transdermal delivery can also be prepared in accordance with the teachings of Lawson et al, “Use of Nanocarriers for Transdermal Vaccine Delivery,” Clin. Pharmacol. Ther. 82(6):641-3 (2007), which is hereby incorporated by reference in its entirety.

The compounds of the present invention may also be administered directly to the airways in the form of an aerosol or mist. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer. Formulations suitable for intranasal nebulization or bronchial aerosolization delivery are also known and can be used in the present invention (see Lu & Hickey, “Pulmonary Vaccine Delivery,” Exp. Rev. Vaccines 6(2):213-226 (2007) and Alpar et al, “Biodegradable Mucoadhesive Particulates for Nasal and Pulmonary Antigen and DNA Delivery,” Adv. Drug Deliv. Rev. 57(3):411-30 (2005), which are hereby incorporated by reference in their entirety.

The pharmaceutical compositions of the present invention can also include an effective amount of a separate adjuvant. Suitable adjuvants for use in the present invention include, without limitation, aluminum hydroxide, aluminum phosphate, aluminum potassium sulfate, beryllium sulfate, silica, kaolin, carbon, water-in-oil emulsions, oil-in-water emulsions, muramyl dipeptide, bacterial endotoxin, lipid, Quil A, and/or non-infective Bordetella pertussis.

The choice of an adjuvant depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, alum, MPL or Incomplete Freund's adjuvant (Jensen et al., “Adjuvant Activity of Incomplete Freund's Adjuvant,” Adv. Drug Deliv. Rev. 32:173-186 (1998), which is hereby incorporated by reference in its entirety) alone or optionally all combinations thereof are suitable for human administration.

Regardless of the form of the composition, such compositions and preparations should contain at least 0.1% of active agent (as described above). The percentage of the active agent in these compositions may, of course, be varied and may conveniently be between about 1% to about 60% of the weight of the single dose. The amount of active agent in such therapeutically useful compositions is such that an effective dosage will be obtained. Preferred compositions according to the present invention are prepared so that single dosage contains between about 0.1 and 500 mg of active agent.

The compositions of the present invention can be administered orally, parenterally, for example, subcutaneously, intravenously, intramuscularly, intraperitoneally, by intranasal instillation, or by application to mucous membranes, such as, that of the nose, throat, and bronchial tubes.

Another aspect of the present invention relates to a method of inhibiting or delaying onset, or reducing the severity of, type 1 diabetes. This method includes administering to an individual having a risk of developing type 1 diabetes an effective amount of: (i) serpinB13 protein or a polypeptide fragment thereof, (ii) a vaccine according to the first aspect of the invention, (iii) an immunogenic conjugate according to the second aspect of the invention, or (iv) a pharmaceutical composition according to the third aspect of the invention, where the administering is effective to induce an anti-serpinB13 antibody response that inhibits or delays onset of type 1 diabetes, or reduces the severity of type 1 diabetes in the individual.

The method may further include assessing the individual's risk of developing diabetes. This assessment may include determining family history of type 1 diabetes, determining a positive status for at least one anti-islet autoantibody (e.g., anti-insulin, anti-GAD, anti-ZnT8, anti-IA2, and/or ICA), determining a positive status for certain HLA hyplotyes (e.g., DR3/DR4), and/or finding abnormal blood glucose levels without clinical picture of diabetes (e.g., abnormal glucose tolerance test (GTT)).

The measurement of anti-serpinB13 alloantibody level may be carried out by immunoassay, e.g., serum binding to immobilized serpinB13, which is inhibited by more than 25% with competitive inhibitor (serpinB13 in solution).

It is contemplated that the individual to be treated in accordance with the present invention can be any mammal, but preferably a human that is known to be susceptible to the development of type 1 diabetes. The method may further comprise assessing the individual's risk of developing diabetes. In certain embodiments, the individual to receive a composition or vaccine of the invention is an adolescent or juvenile, preferably 10 years or younger. In one embodiment, the individual to receive the composition or vaccine of the present invention may be less than 5 years of age or less than 2 years of age.

The administration of the composition or vaccine may be repeated over a period of hours, days, months, or years. When administering is repeated, an additional therapeutic agent may also be administered such as an anti-CD3 monoclonal antibody or binding fragment thereof, an anti-CD20 monoclonal antibody or binding fragment thereof, or a combination thereof. This method may be effective to inhibit onset of type 1 diabetes, delay onset of type 1 diabetes, or reduce the severity of existing type 1 diabetes.

The severity of existing type 1 diabetes is assessed based on serum glucose levels. For example, glycosylated hemoglobulin levels (A1C) and glucose variations may be used to grade the severity of T1D.

This aspect of the invention is carried out in accordance with the aspects described above.

Yet another aspect of the present invention relates to a method of treating an individual for an immune condition. This method includes administering to an individual having an immune condition an effective amount of: (i) serpinB13 protein or a polypeptide fragment thereof, (ii) a vaccine according to the first aspect of the invention, (iii) an immunogenic conjugate according to the second aspect of the invention, or (iv) a pharmaceutical composition according to the third aspect of the invention, where the administering is effective to treat the immune condition or control symptoms thereof, and where the immune condition is selected from the group consisting of psoriasis, hair loss, and ulcers including diabetic food ulceration.

This aspect of the invention is carried out in accordance with the aspects described above.

The model how serpin vaccine may work in T1D is based on studies with a monoclonal antibody against serpinB13. It was discovered that when serpinB13 binds to anti-seprinB13 mAb in vitro, its inhibitory effect decreases and the activity of its target protease increases. Moreover, FACS analysis revealed that the extracellular portion of CD4 in T cells and CD19 in B cells is cleaved from lymphocytes isolated from NOD mice exposed to anti-serpinB13 mAb (this cleavage was inhibited by an E64 protease inhibitor). Based on these findings, and the fact that serpins are protease inhibitors, it is believed that anti-serpin activity partially restores extracellular proteolysis.

One consequence of an elevated extracellular proteolytic activity is impairment of leukocyte function. For example, it was found that NOD mice treated with anti-serpinB13 mAbs (i) have fewer intraislet inflammatory islets, (ii) their T cells secrete reduced levels of diabetogenic cytokines (e.g., interferon gamma), and (iii) their recovery from T1D is accelerated, especially when animals also receive suboptimal dose of anti-CD3 mAb, which has a proven anti-diabetic effect (see Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety).

A second consequence of an elevated proteolytic activity could be an enhanced renewal of insulin-producing cells. Specifically, it was found that treatment of Balb/c mice with anti-serpinB13 mAb in the setting of STZ-induced diabetes enhances expression of genes that have been associated with the differentiation of endocrine cells in the pancreatic islets. It was also found that young Balb/c mice that receive serpinB13 mAb after STZ injection, or serpinB13 itself before STZ injection, develop a less severe form of diabetes and exhibit a faster rate of recovery compared with mice injected with IgG control. Of note, Balb/c animals treated according to the protocol used tend to recover spontaneously from the disease, which makes them well suited for studies of beta cell regeneration.

Thus, the vaccines and pharmaceutical formulations of the present invention should be offered to young children who have an elevated risk for T1D and lack anti-serpin immunity. The rationale for selecting this group of subjects is two-fold. First, the natural autoantibody response against serpinB13 appears to be the strongest early in life when regenerative changes in the pancreatic tissue are preserved. Thus, the therapeutic strategy that is based on stimulating regeneration of the pancreatic tissue should have better rate of succeeding when offered to very young children compared with older children in whom the ability to regenerate pancreatic islets is compromised. Second, it was found that the youngest group of children with T1D (age 0-5 years) shows the lowest output of anti-serpin antibodies (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety); thus this age group should be more sensitive to the effects of anti-serpin therapy compared with other age groups with T1D in which frequency of anti-serpin antibodies is higher.

Having now generally described the invention, the same will be more readily understood through reference to the following examples which are provided by way of illustration, and are not intended to be limiting of the present invention, unless specified.

### EXAMPLES

The Examples set forth below are for illustrative purposes only and are not intended to limit, in any way, the scope of the present invention.

### Materials and Methods for Example 1

Mice. The NOD/LtJ and BDC2.5 TCR transgenic NOD mice were purchased from the Jackson Laboratory (Bar Harbor, Me.) and used to study the effects of treatment with anti-serpinB13 mAb. The University Committee on Animal Resources at the University of Rochester approved all mouse experiments.

Antibodies. FITC-conjugated mAbs were used against CD19 (clone 1D3) and V134 TCR chain (clone KT4); phycoerythrin (PE)-conjugated mAbs were used against B220 (clone RA3-6B2), CD4 (clone RM4-5), TCR (H57-597) and LCA (clone 30-F11) (BD Biosciences), and allophycocyanin (APC)-conjugated mAb was used against IFNγ (clone XMG1.2). A monoclonal antibody against serpinB13 (clone B29) was produced in as described before (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety).

Reagents. Rhodamine 110, bis-(CBZ-L-phenylalanyl-L-arginine amide) was purchased from Life Technologies. E64, an irreversible, potent, and selective inhibitor of cysteine proteases was obtained from Sigma. BDC2.5 mimotope (RTRPLWVRME) was synthesized at the Keck facility of the Yale University School of Medicine. Recombinant mouse CD4 was from Sino Biological Inc. The purified mouse seprinB13 expressed in the baculovius and E. coli were obtained from GeneScript. The purified keratin 19 was from Abcam, Inc.

Treatment of NOD/LtJ mice with anti-serpinB13 mAb and protease inhibitor. Four-week-old female NOD/LtJ mice were injected intravenously 4 times over a period of 10 days with anti-serpinB13 mAb (100 μg/injection). In addition, during the same period some animals were also injected intraperitoneally with the protease inhibitor E64 at 10 mg/kg per day for several consecutive days. Control mice were treated with diluent (a sterilized PBS solution containing 10% dimethylsulfoxide) and control IgG. Twenty-four hours after the last injection, the mice were sacrificed and cells for their lymphoid organs and pancreatic islets were subjected to FACS analysis.

Luminex assay. Luminex-based technology was used to measure the serum-binding activity of serpinB13 exactly as described in Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety.

ELISA. Quantikine Immunoassay (R&D Systems) was used to measure IFN-γ concentration according to the manufacturer's recommendations.

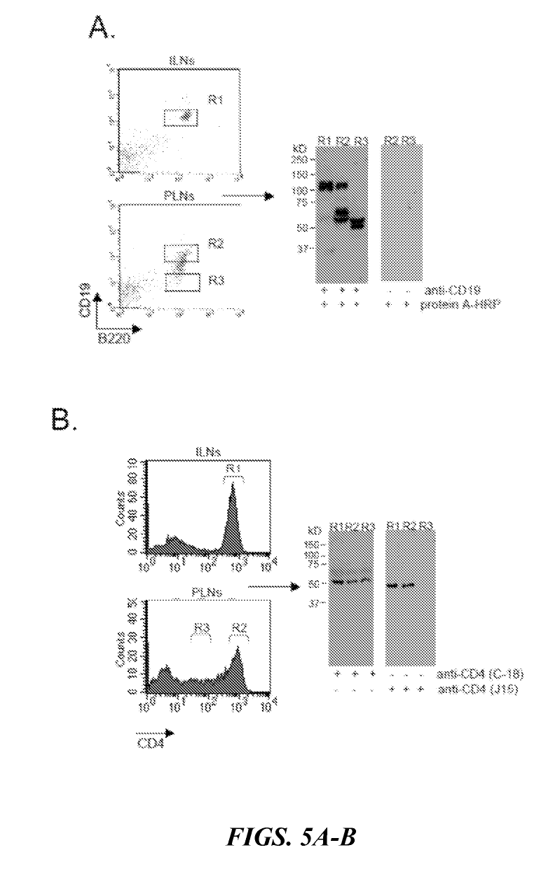

Preparation of CD4 T cells and APCs. CD4 T cells were isolated by sorting as described in the legend to FIG. 5. T-cell depleted antigen presenting cells (APCs) were prepared by Ab-mediated complement lysis of NOD splenocytes. Briefly, spleen cells were depleted of erythrocytes by centrifugation on a lymphocyte separation medium (MP Biomedicals; Solon, Ohio) then incubated, first with a mixture of anti-Thy 1 (Y-19), anti-CD8 (TIB-105), and anti-CD4 (GK1.5) mAbs and then with low-toxicity rabbit complement and 50 μg/ml mitomycin C (Sigma-Aldrich). Purity of the APC was 90% to 95%, as determined by staining with anti-MHC class II mAb.

Immunochemistry. The pancreata were embedded in an optimal cutting temperature (OCT) medium, instantly frozen in a dry-ice/2-methylbutane bath, and then cut into 5-μm sections. To detect glucagon, tissue sections were stained with purified rabbit anti-mouse glucagon IgG (Thermo Fisher Scientific) (1:50) followed by a secondary antibody, Alexa fluor 488 goat anti-rabbit IgG (Invitrogen) (1:200). To detect CD31, FITC-conjugated rat anti-mouse CD31 mAb was used (BD Pharmingen) (1:100). To detect keratin 19, purified rabbit anti-mouse keratin 19 IgG was used at (US Biological) (1:200) and followed by a secondary antibody, Alexa fluor 488 goat anti-rabbit IgG (1:200). To detect serpinB13, purified mouse anti-serpinB13 mAb was used (1:1000, final concentration: 3 μg/mL) followed by a secondary antibody, Alexa fluor 568 goat anti-mouse (Invitrogen) (1:200). The sections were viewed through an immunofluorescence microscope (Nikon Eclipse 50i) fitted with a SPOT digital camera using the SPOT advanced program (Diagnostic Instruments, Inc.). The images were captured with an objective 20×0.5 NA or 10×0.25 NA and processed using Photoshop (7.0 Adobe).

Western blots. To verify the expression of His-tagged proteins, including clade B serpins, 293T-cell transfectants were lysed in a buffer containing 1% NP40, 150 mM NaCl, 50 mM Tris, (pH 7.4), 1 mM Na₃VO₄, 1 mM phenylmethylsulfonyl fluoride and a commercial protease inhibitor (Roche Diagnostics). Protein samples from precleared cell lysates were fractionated under reducing conditions on a 9% sodium dodecyl sulfate-polyacrylamide gel. After electrophoresis, the proteins were electroblotted onto nitrocellulose membranes (Bio-Rad), blocked with 5% nonfat dry milk, and probed with anti-His₍₆₎ rabbit antibody (1 μg/mL) or anti-serpinB13 mAb (1 μg/mL) followed by horseradish peroxidase (HRP)-linked protein A (Sigma) (1:2000) and goat anti-mouse secondary antibody (GE Healthcare) (1:10000), respectively. To determine the level of expression of CD19, B cells were isolated from the PLNs and processed using methods similar to those used with 293T-cell transfectants. The blots were stained with anti-CD 19 (M-20, Santa Cruz) rabbit polyclonal IgG (4 μg/mL) followed by protein A-HRP (1:2000). To determine the level of expression of CD4, T cells were isolated from the PLNs by sorting and lysed as described above. The blots were then stained with 2 different antibodies against CD4: (1) C-18 (Santa Cruz), a goat polyclonal IgG (1:1000), which was followed by HRP-conjugated donkey anti-goat polyclonal IgG (1:1000); and (2) J15 (Santa Cruz) (1:100), a rat monoclonal IgG, followed by goat anti-rat polyclonal IgG (1:10000). The immunoblots were developed using an enhanced chemiluminescence detection system (Amersham Biosciences).

Measurement of substrate cleavage by cathepsin. Cathepsin L (Calbiochem; final dilution: 1:5000) was incubated in an appropriate buffer (50 mM sodium acetate, pH 5.5, 4 mM dithiothreitol [DTT], and 1 mM EDTA) at 25° C. for 45 minutes either alone or in the presence of purified serpinB13 (GeneScript; final concentration: 1.5 μg/mL]). Anti-serpinB13 mAb or IgG control were also added (final concentration: 10 ng/mL) to 2 reaction mixtures. The cleavage by cathepsin L in vitro was determined by adding the fluorescent substrate (CBZ-Phe-Arg)₂-R110 (final concentration: 1 μM) and measuring its rate of hydrolysis over time using a FLUOstar OPTIMA microplate reader. The hydrolysis was followed by measuring the fluorescence of the cleaved substrate every 20 seconds for 20 minutes. The data are presented as fold induction of the peak fluorescence value over background with the substrate alone. To assay cathepsin-mediated hydrolysis in the tissue, the pancreata were digested using the collagenase P/DNAase I (Roche) and homogenized using a VWR pellet mixer (VWR International) in the cell lysis buffer that was supplied with the cathepsin L activity assay kit (BioVision). The lysates were incubated with substrate labeled with amino-4-trifluoromethyl coumarin (AFC). The AFC cleaved by cathepsin L was read using a SynergyMX fluorescence microplate reader (Biotek) at excitation and emission wavelengths of 400 nm and 505 nm. Protein concentration was measured (BCA protein assay) in all samples to normalize that data.

Isolation of islets and pancreatic tissue. Pancreatic islets were isolated using the collagenase/DNAase I digestion method and handpicked under a stereomicroscope. Islet cell suspensions were obtained by treating the islets with Cellstripper buffer (Invitrogen; cat. #25-056-C1) for 5 minutes at 37° C. 100 μL of tissue digest were used to analyze proteases activity in the pancreas.

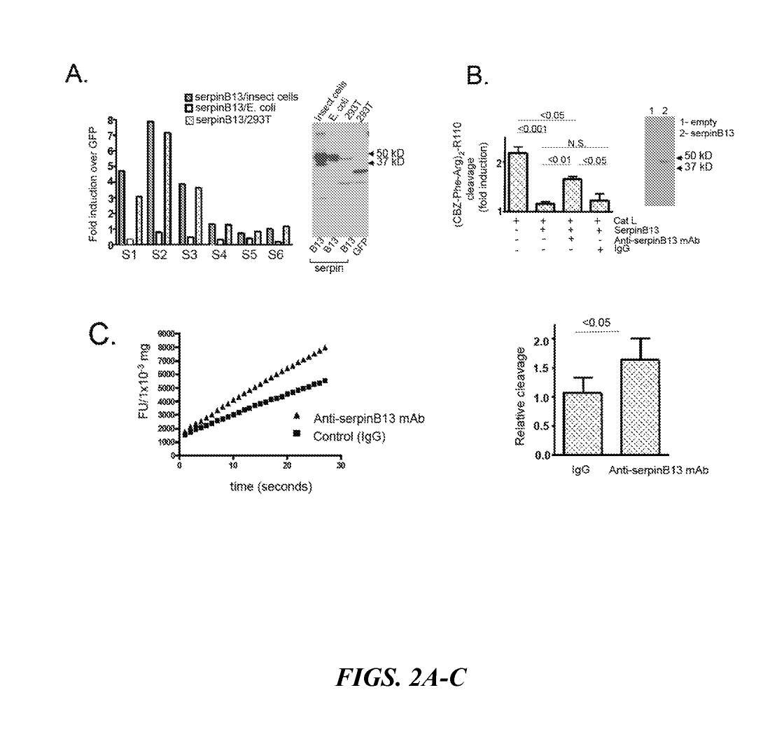

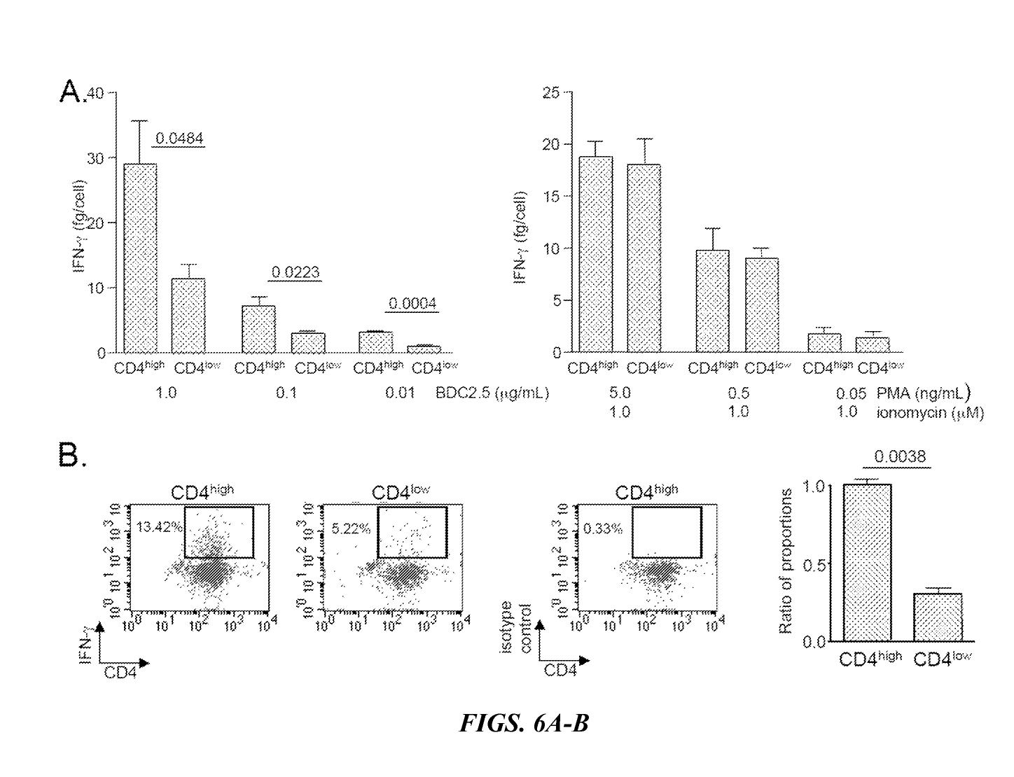

Statistics. Statistical analyses were performed using the T test (FIGS. 2C and 6A-6B) and one-way Anova test (FIGS. 2B, 3A-3B and 4A-4B). A p value less than 0.05 was used to indicate significance. Data are presented as mean±standard deviation.

### Example 1

### Anti-SerpinB13 Autoantibodies Actively Protect from Autoimmune Diabetes

The balance between proteases and their inhibitors is vital to the survival of multicellular organisms (Luke et al., “An Intracellular Serpin Regulates Necrosis by Inhibiting the Induction and Squealae of Lysosomal Injury,” Cell 130:1108-1119 (2007), which is hereby incorporated by reference in its entirety). Enhanced protease activity impacts negatively on homeostasis by up-regulating the cleavage of native proteins into short peptides and increasing their presentation to autoreactive T cells (Casciola-Rosen et al., “Cleavage by Granzyme B is Strongly Predictive of Autoantigen Status. Implications for Initiation of Autoimmunity,” J. Exp. Med. 190:815-825 (1999), which is hereby incorporated by reference in its entirety). Proteases can also influence many other processes including tissue remodeling and resolution of inflammation (Yargoni et al., “Prevention of Murine EAE by Oral Hydrolytic Enzyme Treatment,” J. Autoimmunity 12:191-198 (1999); Wiest-Ladenburger et al., “Protease Treatment Delays Diabetes Onset in Diabetes-Prone Nonobese Diabetic (NOD) Mice,” Int. J. Immunotherapy 13:75-78 (1997), which are hereby incorporated by reference in their entirety) suggesting that their activity is not always pathogenic. Given the critical role of protease activity during formation of autoimmune inflammation (Casciola-Rosen et al., “Cleavage by Granzyme B is Strongly Predictive of Autoantigen Status. Implications for Initiation of Autoimmunity,” J. Exp. Med. 190:815-825 (1999); Asagiri et al., “Cathepsin K-Dependent Toll-Like Receptor 9 Signaling Revealed in Experimental Arthritis,” Science 319:624-627 (2008); Reinhold et al., “DP1V/CD26, APN/CD13 and Related Enzymes as Regulators of T cell Immunity: Implication for Experimental Encephalomyelitis and Multiple Sclerosis,” Frontiers Biosci. 13:2356-2363 (2008), all of which are hereby incorporated by reference in their entirety), it is not surprising that they are modulated by a number of inhibitors including B-clade molecules, also known as (ov)-serpins (Irving et al., “Phylogeny of the Serpin Superfamily: Implications of Patterns of Amino Acid Sequences for Structure and Function,” Genome Res. 10:1845-1864 (2000); Silverman et al., “Human Clade B Serpins (ov-serpins) Belong to a Cohort of Evolutionary Dispersed Intracellular Proteinase Inhibitor Clades That Protect Cells From Promiscuous Proteolysis,” Cell. Mol. Life Sci. 61:301-325 (2004), both of which are hereby incorporated by reference in their entirety). The inhibitory activity of these serpins may in turn be regulated. For example, the cofactor, heparin, markedly enhances the ability of ov-serspins SCCA-1 and SCCA-2, to neutralize their target protease (Higgins et al., “Heparin Enhances Serpin Inhibition of the Cysteine Protease Cathepsin L.,” J. Biol. Chem. 285:3722-3729 (2010), which is hereby incorporated by reference in its entirety).

Recently it was found that young diabetes-prone NOD mice (Anderson et al., “The NOD Mouse: A Model of Immune Dysregulation,” Ann. Rev. Immunol. 23:447-485 (2005); Solomon et al., “The Pathogenesis of Diabetes in the NOD Mouse,” Adv. Immunol. 84:239-264 (2004), both of which are hereby incorporated by reference in their entirety) secrete autoantibodies against a member of Clade B family called serpinB13 (Nkashima et al., “Genomic Cloning, Mapping, Structure and Promoter Analysis of HEADPIN, A Serpin Which is Down-Regulated in Head and Neck Cancer Cells,” Biochim. Biophys. Acta 1492:441-446 (2000); Welss et al., “Hurpin is a Selective Inhibitor of Lysosomal Cathepsin L and Protects Keratinocytes From Ultraviolet-Induced Apoptosis,” Biochemistry 42:7381-7389 (2003); Jayakumar et al., “Inhibition of the Cysteine Proteinases Cathepsins K and L by the Serpin Headpin (SERPINB13): A Kinetic Analysis,” Arch. Biochem. Biophys. 409:367-374 (2003), all of which are hereby incorporated by reference in their entirety) and that this response is associated with protection from early-onset autoimmune diabetes (Czyzyk et al., “Enhanced Anti-Serpin Antibody Activity Inhibits Autoimmune Inflammation in Type 1 Diabetes,” J. Immunol. 188:6319-6327 (2012), which are hereby incorporated by reference in its entirety). Because autoantibodies that interfere with enzyme cascade activities have been described in several pathologic conditions (Spitzer, R. E., “Serum C3 Lytic System in Patients with Glomerulonephritis,” Science 164:436-437 (1969); Gawryl et al., “Inactivation of Factor VIII Coagulant Activity by Two Different Types of Human Antibodies,” Blood 60:1103-1109 (1982); Alsenz et al., “Autoantibody-Mediated Acquired Deficiency of C1 Inhibitor,” N. Eng. J. Med. 316:1360-1366 (1987); Jackson et al., “An IgG Autoantibody, Which Inactivates C1-Inhibitor,” Nature 323:722-724 (1986), all of which are hereby incorporated by reference in their entirety) whether anti-serpinB13 autoantibodies actively protect from autoimmune diabetes was addressed by regulating the balance between this serpin and its protease targets. Using a monoclonal antibody against serpinB13 as a model, it was found that humoral activity against this serpin stimulates protease activity and causes enhanced cleavage of lymphocyte surface molecules. The present data also shows that it is likely that natural anti-serpin autoantibodies act in a similar fashion to that described for monoclonal antibody during months preceding the development of autoimmune diabetes. Ultimately, this response may interfere with normal function of inflammatory cells in the pancreatic tissue and contribute to slower progression of pathologic changes in autoimmune diabetes.

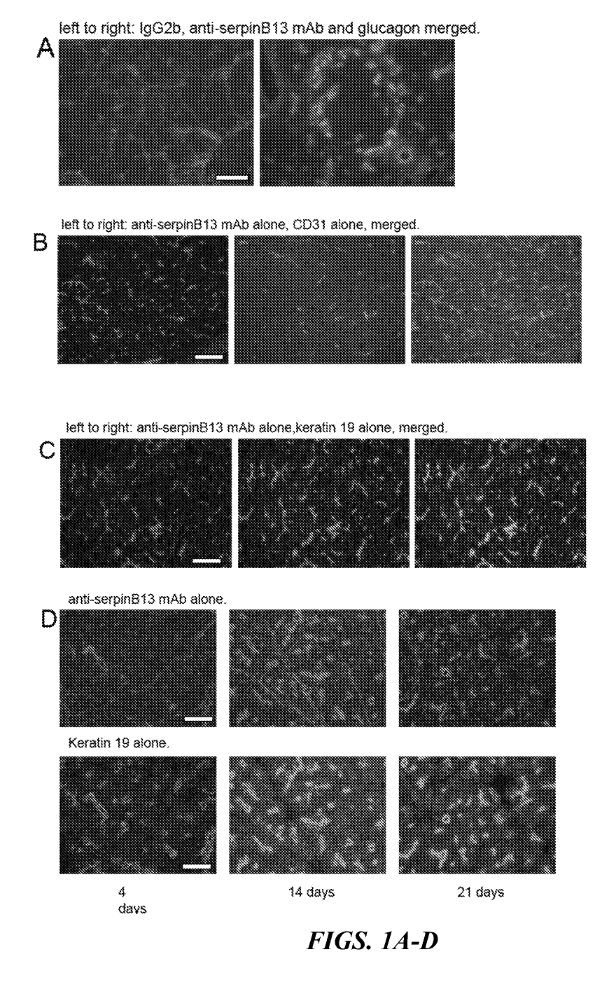

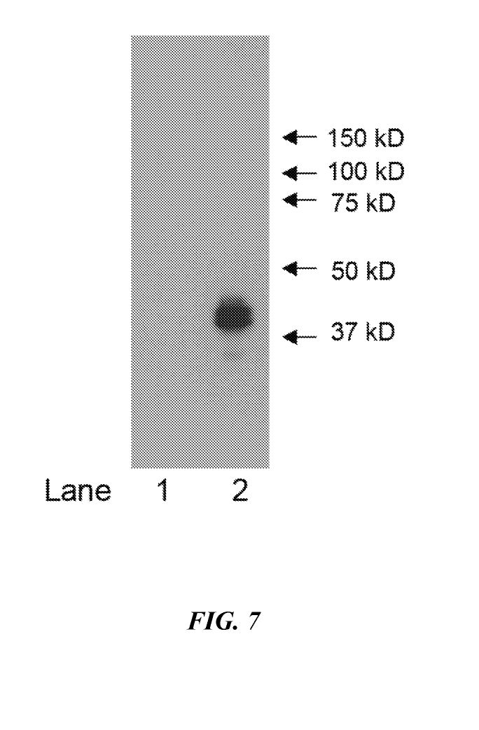

SerpinB13 is expressed in the pancreas. Previous analysis revealed that serpinB13 is expressed in the pancreas, although the exact tissue compartment in which it is likely to be found remained unclear (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety). To identify this compartment frozen sections of NOD pancreatic tissue were stained using a monoclonal antibody (mAb) that was produced in the laboratory (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety). It was found that a small amount of serpinB13 is expressed in the pancreatic islets (FIG. 1A) but a much larger amount is present in the exocrine pancreas. Specifically, keratin 19-positive ductal cells were more deeply stained with anti-serpinB13 mAb compared with the acini (FIGS. 1A-1C). Of note, this pattern of staining did not appear to be due to the cross-reactivity of the anti-serpin monoclonal antibody, because it failed to stain purified keratin19 (FIG. 7). A strong induction of serpinB13 in the pancreas was observed during the first few weeks of life (FIG. 1D). This early expression pattern may be responsible, in part, for the generation of a humoral response to serpinB13 in young NOD mice (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety).

Anti-serpinB13 mAb influences protease(s) in the pancreatic tissue. It was hypothesized that anti-serpinB13 autoantibody might influence the development of pathologic changes in the islets directly by binding to serpinB13 and by changing the ability of proteases to cleave their substrates. Initial support for this hypothesis was provided through the finding that the serpinB13 produced in 293T cells or in insect cells was a better target for autoantibodies than the serpinB13 purified from bacteria (FIG. 2A). This suggests that anti-serpinB13 autoantibodies mainly recognize properly folded or posttranslationally modified epitopes and, thus, may neutralize the inhibitory effect of ov-serpins on their protease targets. Consistent with this finding, it was determined that the anti-serpinB13 mAb partially prevents cathepsin L-mediated cleavage of the fluorescent substrate (CBZ-Phe-Arg)₂-R110 from inhibition by serpinB13 in vitro (FIG. 2B).

To gain additional insight into the effect of anti-serpin activity on proteases in vivo, young NOD mice were injected with anti-serpinB13 mAb and the function of cathepsin L in pancreatic tissue was analyzed. Consistently with the in vitro observations, it was found that cathepsin L was enhanced in the pancreas of mice that received anti-serpinB13 mAb (FIG. 2C).

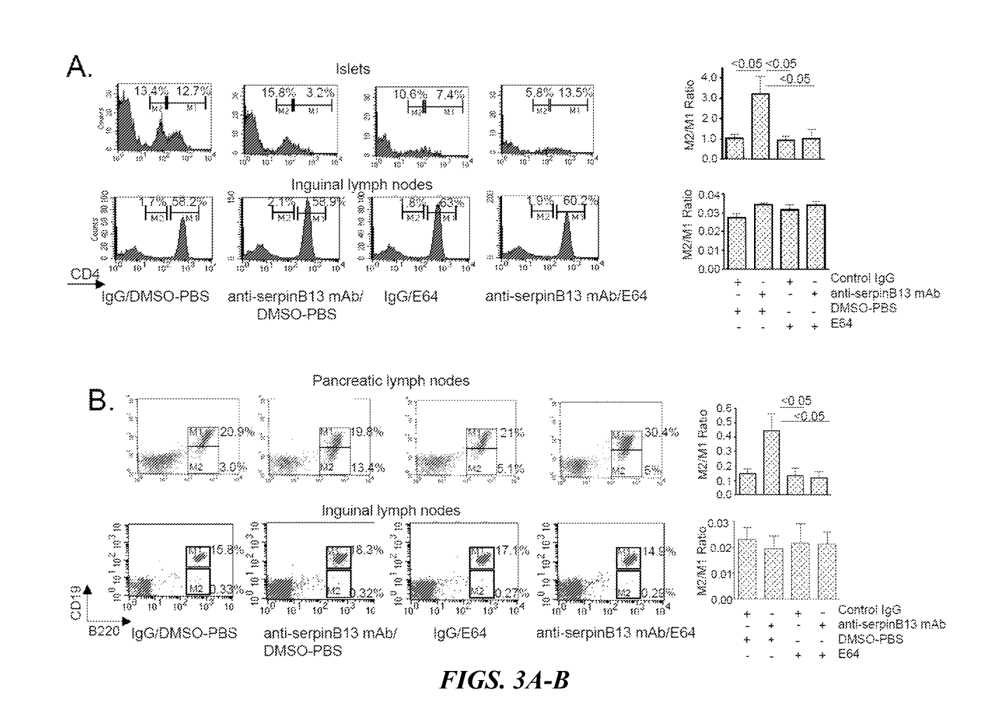

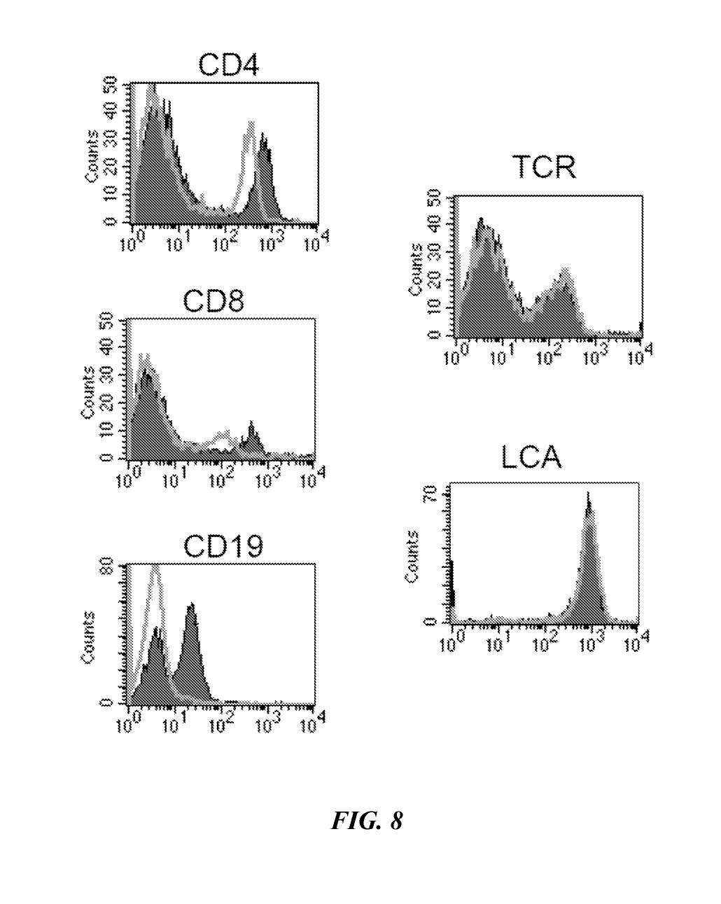

Both monoclonal antibody and natural autoantibodies against serpinB13 upregulate the cleavage of lymphocyte surface molecules in the pancreas. It is possible that enhanced proteolysis results in injury to leukocytes accumulating in the endocrine compartment of the pancreas and helps limit the inflammatory response in that compartment. Therefore, markers of antibody-enhanced proteolysis were sought that could explain reduced inflammation. The initial in vitro screen revealed that exposure of mouse splenocytes to cathepsin L results in a partial or complete loss (due to the cleavage) of several important surface lymphocyte markers, including CD4 and CD19, but has only a minimal effect on other surface molecules (FIG. 8). This provided a rationale to study the expression profiles of CD4 and CD19 in greater detail in NOD mice treated with anti-serpinB13 mAb. It was found that a broad spectrum of expression of CD4 and CD 19 exists in both the islets and pancreatic lymph nodes (PLNs) and that anti-serpinB13 mAb exposure caused a significant shift that favors cells expressing low to intermediate amounts of these markers. However, this shift was abolished in animals that received both anti-serpinB13 mAb and the protease inhibitor E64 (FIGS. 3A and 3B). These changes were observed in the pancreas and PLNs but not in the distant lymphoid organs (e.g., inguinal lymph nodes).

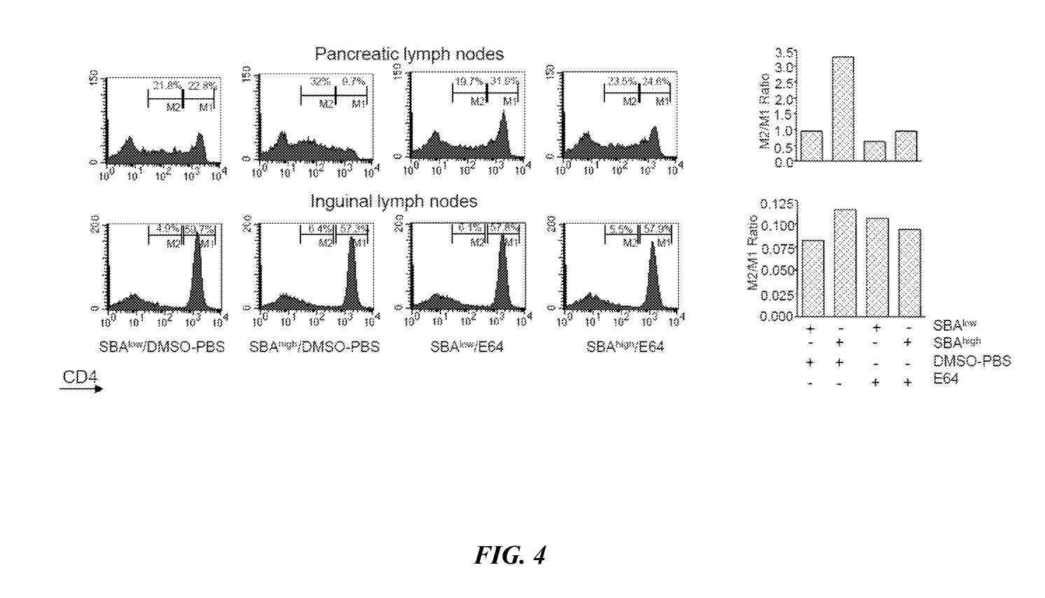

It is possible that the effects of anti-serpinB13 mAb do not reflect the effects of elevated levels of natural anti-serpinB13 autoantibodies exactly. To address this problem, NOD mice were compared with distinct levels of anti-serpinB13 autoantibodies. It was found that young animals with high levels of endogenous anti-serpinB13 autoantibodies (SBA^{high}) have a markedly reduced population of islet-associated CD4^{high} cells compared with animals with low levels of these autoantibodies (SBA^{low}). The number of islet-associated CD4^{high} cells did not decrease, however, in SBA^{high} mice that received the protease inhibitor E64 (FIG. 4). This observation suggests that natural anti-serpin autoantibodies regulate proteases in vivo in a fashion similar to that described for the anti-serpinB13 mAb.

To verify that the considerably reduced levels of CD19 on the cell surface is a sign of degradation, a Western blot analysis was performed of CD19 in B220⁺/CD19⁺ B cells isolated from inguinal lymph nodes and from PLN B cells expressing either high or low amounts of CD19. The B220⁺/CD19⁺ B cells isolated from the inguinal lymph nodes (R1) only expressed the nondegraded form of CD19, whereas the B220⁺/CD19⁺ B cells isolated from the PLNs (R2) contained both the degraded and nondegraded (cleaved) form of CD 19. The B220⁺/CD19^{low} cells from the PLNs (R3) had no intact CD19 molecules; in fact, CD19 molecules were quantitatively and qualitatively more severely degraded in the B220⁺/CD19^{low} cells compared with the B220+/CD19^{high} cells (FIG. 5A). T cells were also isolated from the PLNs based on their limited expression of CD4 (R3). These cells underwent Western blot analysis then stained with one antibody that recognizes an extracellular domain of CD4 and another that recognizes an intracellular domain of CD4 (FIG. 5B). The extracellular domain was not detected in the isolated T cells (R3), but the stain for the intracellular domain was positive, indicating that these cells do express CD4. Thus, these findings demonstrate that B220⁺/CD19^{low} B cells and CD4^{low} T cells accumulate in the PLNs and pancreatic islets in response to anti-serpinB13-induced augmentation of proteases and increased cleavage of the extracellular portion of CD19 and CD4.

T cells with the cleaved form of CD4 secrete less IFN-γ compared with T cells expressing intact CD4 molecule. To determine significance of changes that are caused by anti-serpinB13 antibodies in lymphocytes, CD4^{low} and CD4^{high} T cells were isolated from the PLNs of BDC2.5 TCR transgenic NOD mice and compared these two cell populations for their ability to secrete cytokines. The focus was on IFN-γ because this cytokine was heavily implicated in the pathogenesis of T1D (Sarvetnick et al., “Insulin-Dependent Diabetes Mellitus Induced in Transgenic Mice by Ectopic Expression of Class II MHC and Interferon-γ,” Cell 52:773-782 (1988), which is hereby incorporated by reference in its entirety). It was found that both during the primary and secondary stimulation with an antigenic peptide, T cells with the truncated form of CD4 produce significantly less IFN-γ compared with T cells expressing normal levels of CD4 molecule (FIGS. 6A left, and 6B). This defect was not observed during stimulation, which does not require CD4 (FIG. 6A, right).

The existence of a novel antibody that recognizes and inhibits the protease inhibitor serpinB13 in the setting of autoimmune diabetes has been demonstrated by way of the present invention. This antibody leads to marked histological changes in the composition of the inflammatory infiltrate in the pancreatic islets of NOD mice, and ultimately contributes to a better clinical outcome (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety). In the present study, it was found that serpinB13 is expressed mainly in the exocrine portion of the pancreas, most notably in the epithelial lining of the pancreatic ducts. This pattern of expression suggests that the protease targets of serpinB13 (e. g. cathepsin L and K) are also expressed in this tissue compartment. Although this possibility was not addressed in this study, it is supported by reports from other studies in which the expression of cathepsin K was detected in the bronchial and bile duct epithelial cells and in the urothelia (Buhling et al.,“Expression of Cathepsin K in Lung Epithelial Cells,” Am. J. Respir. Cell Mol. Biol. 20:612-619 (1999); Haeckel et al., “Expression of Cathepsin K in the Human Embryo and Fetus,” Dev. Dyn. 216:89-95 (1999), both of which are hereby incorporated by reference in their entirety).

The features of the protective mechanism of anti-serpin antibody are the inhibition of serpinB13 and consequent maintenance of limited function of its protease targets, which in turn, facilitate the cleavage of cell-surface molecules expressed in lymphocytes including extracellular domains of CD4 and CD19. It is likely that other molecules are also cleaved by anti-serpinB13-enhanced proteases. Consistent with this hypothesis are other studies in which the investigators demonstrated that (1) administration of exogenous hydrolytic enzymes can lead to a partial resolution of inflammation (Yargoni et al., “Prevention of Murine EAE by Oral Hydrolytic Enzyme Treatment,” J. Autoimmunity 12:191-198 (1999); Wiest-Ladenburger et al., “Protease Treatment Delays Diabetes Onset in Diabetes-Prone Nonobese Diabetic (NOD) Mice,” Int. J. Immunotherapy 13:75-78 (1997), both of which are hereby incorporated by reference in their entirety), (2) proteolytic enzymes can impair the function of many different cell-surface molecules expressed in inflammatory cells (Roep et al., “Modulation of Autoimmunity to Beta-Cell Antigens by Proteases,” Diabetologia 45:686-692 (2002), which is hereby incorporated by reference in its entirety, and (3) the expression of CD25 in the Treg cells is markedly reduced at the site of inflammation (Tang et al., “Central Role of Defective Interelukin-2 Production in the Triggering of Islet Autoimmune Destruction,” Immunity 28:687-697 (2008); Lazarski et al., “Regulating Treg Cells at Sites of Inflammation,” Immunity 29:511 (2008), both of which are hereby incorporated by reference in their entirety). Of note, other autoantibodies have been found to be associated with protection from T1D (She et al., “Heterofile Antibodies Segregate in Families and are Associated With Protection From Type 1 Diabetes,” Proc. Natl. Acad. Sci. USA 96:8116-8119 (1999); Menard et al., “Anti-GAD Monoclonal Antibody Delays the Onset of Diabetes Mellitus in NOD Mice,” Pharmac. Res. 16:1059-1066 (1999); Koczwara et al., “Transmission of Maternal Islet Antibodies and Risk of Autoimmne Diabetes in Offspring of Mothers with Type 1 Diabetes,” Diabetes 53:1-4 (2004), all of which are hereby incorporated by reference in their entirety), although the exact molecular events following their mode of action have not been determined

It should be noted that anti-clade B serpin autoantibodies may slow down development of the autoimmune from of diabetes by other mechanisms. For example, an anti-serpin autoantibody-mediated upregulation of proteases may lead to pancreatic islet tissue injury followed by the compensatory regeneration of islet tissue, de novo formation of islets, or both (Miralles et al., “TGF-β Plays a Key Role in Morphogenesis of the Pancreatic Islets of Langerhans by Controlling the Activity of the Matrix Metalloproteinase MMP-2,” J. Cell. Biol. 143:827-836 (1998), which is hereby incorporated by reference in its entirety). Support for this hypothesis has been provided through the observation that cathepsin K (Boyce et al., “Future Anti-Catabolic Therapeutic Targets in Bone Disease.,” Ann. New York Acad. Sci. USA 106:447-457 (2006), which is herby incorporated by reference in its entirety) and cathepsin L (Afonso et al., “The Expression and Function of Cystatin C and Cathepsin B and Cathepsin L During Mouse Embryo Implantation and Placentation,” Development 124:3415-3425 (1997); Robker, R. L. (2000) Progesterone-related genes in the ovulation process: ADAMTS-1 and cathepsin L proteases. Proc. Natl. Acad. Sci. USA 97, 4689-4694 (2000), all of which are hereby incorporated by reference in their entirety) play important roles in tissue remodeling and that the expression of transcription factors associated with the differentiation of pancreatic islets is increased in NOD mice treated with anti-serpinB13 mAb. Another possibility for protective mechanisms that are delivered by anti-serpin antibodies may involve (1) induction of neonatal beta cell apoptosis and immunological tolerance to molecules released from dying cells (Hugues et al., “Tolerance to Islet Antigens and Prevention From Diabetes Induced by Limited Apoptosis of Pancreatic β cells,” Immunity 16:169-181 (2002), which is hereby incorporated by reference in its entirety); and (2) generation of the biologically active form of transforming growth factor-β, which is responsible for generating regulatory T cells with anti-inflammatory properties (Pesu et al., “TGF T-Cell-Expressed Proprotein Convertase Furin is Essential for Maintenance of Peripheral Immune Tolerance,” Nature 455:246-250 (2008), which is hereby incorporated by reference in its entirety).

In addition to evidence presented in this study that the preservation of proteases in the pancreas helps to keep inflammation in check and in another study indicating that the cathepsin L gene belongs to a group of the 100 “protective genes” in NOD mice (Fu et al., “Early Window of Diabetes Determinism in NOD Mice, Dependent on the Complement Receptor CRIg, Identified by Noninvasive Imaging,” Nature Immunol. 13:361-368 (2012), which is hereby incorporated by reference in its entirety), it has been demonstrated that down-regulation of proteases using chemical inhibitors (Ishimaru et al., “Critical Role of Cathepsin-Inhibitors for Autoantigen Processing and Autoimmunity,” Adv. Enzyme Reg. 44:309-320 (2004); Yamada et al., “Cathepsin L Inhibition Prevents Murine Autoimmune Diabetes Via Suppression of CD8⁺ T Cell Activity,” PloS One 5:e12894 (2010), which are hereby incorporated by reference in their entirety), genetic knockout techniques (Maehr et al., “Cathepsin L is Essential for Onset of Autoimmune Diabetes in NOD Mice,” J. Clin. Invest. 115:2934-2943 (2005); Hsing et al., “Roles of Cathepsin S, L, and B in Insulitis and Diabetes in the NOD Mouse, “J. Autoimmunity 34:96-104 (2010), both of which are hereby incorporated by reference in their entirety) or natural serpin inhibitors (Lu et al., “α1-antitrypsin Gene Therapy Modulates Cellular Immunity and Efficiently Prevents Type 1 Diabetes in Nonobese Diabetic Mice,” Hum. Gene Ther. 17:625-634 (2006); Koulmanda et al., “Curative and Cell Regenerative Effects of α1-Antitrypsin Treatment in Autoimmune Diabetic NOD Mice,” Proc. Natl. Acad. Sci. USA 105:16242-16247 (2008), both of which are hereby incorporated by reference in their entireties) can block inflammation in the pancreatic islets of NOD mice. By using a monoclonal antibody against serpinB13, the role of extracellular proteases in the regulation of inflammation was examined, despite the fact that protease targets of serpinB13 (e.g., cathepsin L and K) are mainly located in the lysosomal and endosomal vesicles. There is ample evidence that these cathepsins can be secreted to process proteins in the extracellular matrix where they can promote tissue remodeling (Reiser et al., “Specialized Roles for Cysteine Cathepsins in Health and Disease,” J. Clin. Invest. 120:3421-3431 (2010); Dickinson et al., “Cysteine Peptidases of Mammal: Their Biological Roles and Potential Effects in the Oral Cavity and Other Tissues in Health and Disease,” Crit. Rev. Oral Biol. Med. 13:238-275 (2002), both of which are hereby incorporated by reference in their entirety). Cathepsin L can be active at a close-to-neutral pH, and both hypoxia and acidification that are associated with inflammation can increase the stability of proteases. By contrast, the proteases manipulated in other studies may have been both extracellular and intracellular (Ishimaru et al., “Critical Role of Cathepsin-Inhibitors for Autoantigen Processing and Autoimmunity,” Adv. Enzyme Reg. 44:309-320 (2004); Yamada et al., “Cathepsin L Inhibition Prevents Murine Autoimmune Diabetes Via Suppression of CD8⁺ T Cell Activity,” PloS One 5:e12894 (2010), both of which are hereby incorporated by reference in their entirety), and the manipulation of those proteases may have affected multiple tissues over extended periods of time (Maehr et al., “Cathepsin L is Essential for Onset of Autoimmune Diabetes in NOD Mice,” J. Clin. Invest. 115:2934-2943 (2005); Hsing et al., “Roles of Cathepsin S, L, and B in Insulitis and Diabetes in the NOD Mouse, “J Autoimmunity 34:96-104 (2010), both of which are hereby incorporated by reference in their entirety). As for alpha 1 antitrypsin (Lu et al., “α1-antitrypsin Gene Therapy Modulates Cellular Immunity and Efficiently Prevents Type 1 Diabetes in Nonobese Diabetic Mice,” Hum. Gene Ther. 17:625-634 (2006); Koulmanda et al., “Curative and β Cell Regenerative Effects of α1-Antitrypsin Treatment in Autoimmune Diabetic NOD Mice,” Proc. Natl. Acad. Sci. USA 105:16242-16247 (2008), which is hereby incorporated by reference in its entirety), this serpin neutralizes proteases other than those regulated by serpinB13; thus, its anti-diabetic effect may reflect the inhibition of additional proteases.

In conclusion, anti-clade B serpin antibodies induced under inflammatory conditions can fine-tune the balance between proteases and their inhibitors in damaged tissue and, thus, may contribute to homeostatic events that subdue the early stages of inflammation. If so, the protocols designed to enhance humoral immunity against clade B serpins should impede the progression of pathologic changes that occur in autoimmune diabetes and in other forms of inflammatory disease.

### Example 2

### Efficacy of Anti-SerpinB13 mAb in Stimulating the Regeneration of Insulin Producing Cells

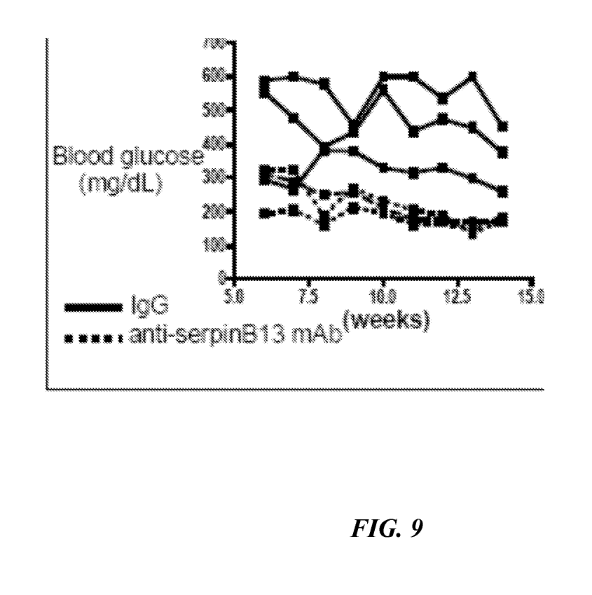

Young Balb/c mice that receive anti-serpinB13 mAb after STZ injection develop a less severe form of diabetes and exhibit a faster rate of recovery compared with mice receiving IgG control (FIG. 9).

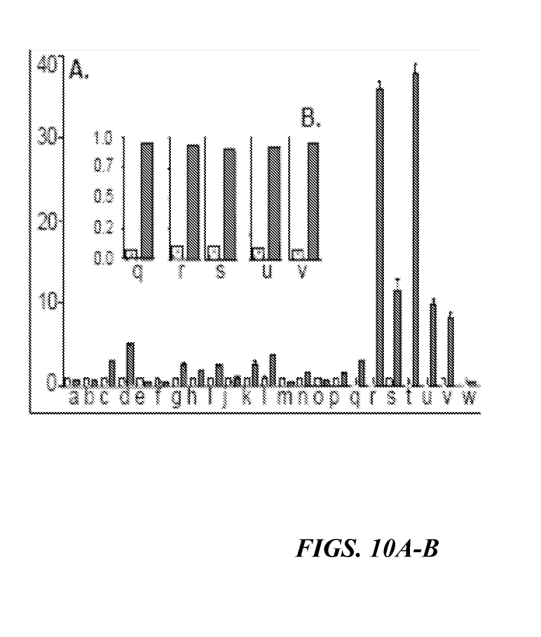

They also exhibit increased expression of islet Reg genes (Terazano et al., “A Novel Gene Activated in Regenerating Islets,” J. Biol. Chem. 263:2111-2114 (1988), which is hereby incorporated by reference in its entirety), which is also seen in NOD mice with elevated endogenous anti-serpinB13 AA levels (FIGS. 10A-B).

These observations suggest that anti-serpin AAs regulate the regeneration of pancreatic endocrine tissue without using anti-inflammatory mechanism. This possibility will be explored in male Balb/c mice injected with STZ 5 to 10 days postpartum (under this treatment, these animals tend to recover spontaneously from the disease by week 20 and, thus, are well suited for studies of beta cell regeneration (Hartmann et al., “Spontaneous Recovery of Streptozotocin Diabetes in Mice,” Exp. Clin. Endocrinol. 93:225-230 (1989), which is hereby incorporated by reference in its entirety) by monitoring beta cell proliferation by FACS analysis and by counting insulin-positive cells that co-stain with anti-Ki67 or anti-Brdu antibodies (the latter will be added to the animal's drinking water) and islet size by histological analysis of frozen pancreatic sections from multiple levels of the pancreas stained with anti-insulin antibody. The proliferation rate will also be determined for islet stem cells that are positive for CD29, CD105, and Sca1 and negative for CD31 and CD45 (approximately 1% of islet cells display this phenotype). The influence of the anti-serpin antibody response on the upregulation of genes associated with beta cell generation and survival will also be explored. Specifically, quantitative PCR analysis will be used to monitor the expression of genes that (1) drive cells toward the endocrine lineage (e.g., Ngn3, insulinomal, and NeuroD1/β1); (2) act as beta cell differentiation factors (Pdx1, Pax4, NeuroD1/β2, MafA, Nkx6.1, and Nkx2.2), (3) help regulate the expression of insulin (Pdx1, MafA, β2, and Nkx2.2); and (4) participate in beta cell proliferation in the adult pancreas (Pax4). Finally, the influence of protease activity will be investigated on the effect of anti-serpinB13 antibodies on islet regeneration in Balb/c mice with STZ-induced diabetes that also lack cathepsin L (a protease target of serpinB13). This will allow a determination of whether the absence of cathepsin L activity compromises the ability of anti-antiserpinB13 mAb to improve the rate of recovery from disease.

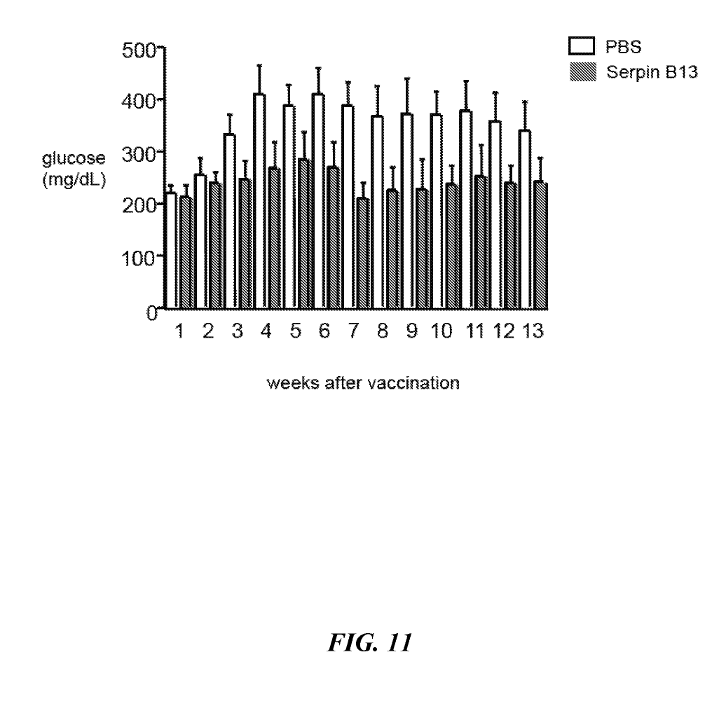

Optimize the protocol of therapy with serpinB13. It is possible that regenerative changes observed in the pancreatic tissue after anti-serpinB13 mAb administration could also be induced by an increase in endogenous antibody level in response to immunization with serpin. Partial support for this hypothesis has been provided by serpinB13-associated protection against severe hyperglycemia in mice (FIG. 11). It will be determined whether optimal serpinB13 treatment affects the islets in a manner similar to that seen with anti-serpinB13 mAbs.

This study will be designed to determine (1) the optimal serpinB13 concentration for preventing STZ-induced diabetes; (2) whether multiple injections of serpinB13 produce a better clinical outcome than a single injection; and (3) whether short serpinB13-derived peptides are as effective as full length serpinB13 in both NOD mice (which resemble autoimmune diabetes in humans (Solomon et al., “The Pathogenesis of Diabetes in the NOD Mouse,” Adv. Immunol. 84:239-264 (2004); Anderson et al., “The NOD Mouse: A Model of Immune Dysregulation,” Ann. Rev. Immunol. 23:447-485 (2005), both of which are hereby incorporated by reference in their entirety)) and mice with STZ-induced diabetes (which causes a limited inflammatory islet response). The responses will be determined by monitoring (1) blood glucose levels; (2) IgM and IgG anti-serpin antibodies at baseline and after immunization using the luminex-based methodology described in Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety; (3) the expression of islet genes using PCR; and (4) the rate of beta-cell proliferation using FACS analysis.

One may argue that the therapeutic effect of serpinB13 in Balb/c mice with STZ-induced diabetes is caused by inhibition of proteases that normally are regulated by this serpinB13 rather than the antiserpinB13 immune response (although the observation that glycemia is reduced several weeks rather than several days after serpinB13 administration tends to support the latter). Therefore, the therapeutic differences between immunocompetent and immunocompromised mice were evaluated. Balb/c mice with severe combined immunodeficiency (“SCID”) will serve as the immunocompromised model.

Examine autoantibodies against SERPINB13 in humans at risk for T1D. Increased secretion of anti-serpinB13 AAs in NOD mice is associated with increased expression of islet Reg genes (FIG. 10) and protection from early-onset diabetes (age<16 weeks) diabetes (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety). In humans, early-onset (age<5 years) T1D has been associated with low output of anti-serpinB13 AAs (Table 1; Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety)). Together these findings suggest an inverse relationship between the anti-serpinB13 AA response and the development of clinical diabetes. These data also suggest that anti-serpin AA levels indirectly reflect the rate of islet renewal.

TABLE 1
Analysis of Anti-serpinB13 AAs (SBA) in
Patients with Recent Onset T1D

Controls

| | |
|---|---|
| SBA Pos | Diabetic Subjects |

| Age | N | (%) | N | SBA Pos (%) |
|---|---|---|---|---|
| 0-5 | 7 | 71% | 6 | 17% |
| 6-10 | 19 | 37% | 21 | 24% |
| 11-15 | 9 | 33% | 16 | 44% |
| 16-20 | 8 | 22% | 12 | 50% |

| | | |
|---|---|---|
| Trend P* | 0.07 | 0.02 |

To determine whether the risk for T1D in humans is associated with secretion of anti-serpinB13 AAs, the rate of anti-serpin AA secretion in individuals who were stratified to have high (>50%, i.e., individuals who are IA-2 AA positive) will be compared to those with low (<10%, i.e., IA-2 AA negative subjects but positive for other pancreatic AAs) risk of developing T1D within 5 years (Decochez et al., “Belgian Diabetes Registry, Combined Positivity for HLA DQ2/DQ8 and IA-2 Antibodies Defined Population at High Risk of Developing Type 1 Diabetes,” Diabetologia 48:687-694 (2005); Decochez et al., “Belgian Diabetes Registry. IA-2 Autoantibodies Predict Impending Type 1 Diabetes in Siblings of Patients,” Diabetologia 45:1658-1666 (2002), both of which are hereby incorporated by reference in their entirety). To do so, serum samples will be examined that are obtained from individuals recruited by the Belgian Diabetes Registry (BDR). Luminex assay will be used to detect anti-serpin AAs (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety).

### Example 3

### Improved Response to Immunization with SerpinB13 and Alum Adjuvant on STZ-Induced Diabetes in Balb/c Mice

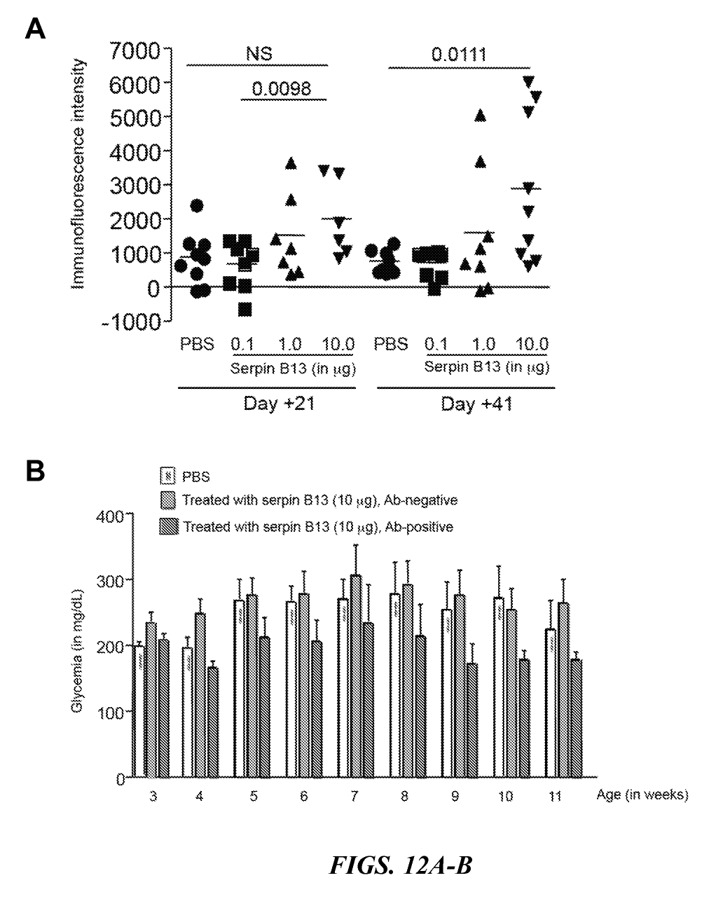

The effect of immunization with serpin B13 on streptozotocin-induced diabetes in Balb/c mice was studied (FIGS. 12A-12B). Results of secretion of anti-serpin antibodies after immunization of mice with different doses of antigen are shown in FIG. 12A. FIG. 12B shows blood glucose levels in mice productively immunized with serpin B12 (red). Animals immunized but failing to secrete antibodies demonstrated similar glucose levels to control animals treated with PBS. Balb/c newborn male mice were injected on Day +5 and +10 with PBS (control) or 0.1, 1.0 or 10 micrograms of purified mouse serpin B13 (GenScript), as indicated. Mice were then screened for the presence of anti-serpin immunoglobulins using Luminex assay (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety). The assay was performed on serum samples collected on Day +21 and Day +46. Blood glucose levels in mice productively immunized with serpin B13 (red) are shown in FIG. 12B. Balb/c male mice were treated with serpin B13 exactly as described in A, and then injected with a single dose of STZ (100 mg/dL) and weakly monitored for blood glucose levels. These animals were also bled on Day +21 and +46 to monitor anti-serpin antibodies as a readout of immunization Animals immunized but failing to secrete antibodies (orange) demonstrated similar glucose levels to control animals treated with PBS.

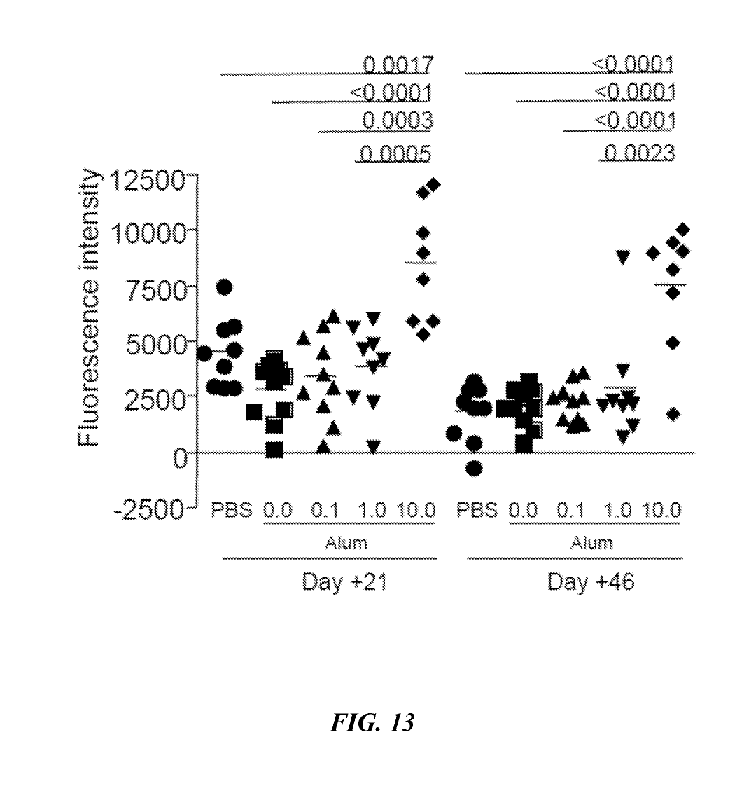

Mice showed an improved response to immunization with serpin B13 and alum adjuvant (FIG. 13). Balb/c newborn male mice were injected on Day +5 and +10 with 100 micrioliters of alum adjuvant or a combination of alum and 0.1, 1.0 or 10 micrograms of purified mouse serpin B13 (GenScript), as indicated. The control mice received PBS. Mice were then screened for the presence of anti-serpin immunoglobulins using Luminex assay, exactly as described in Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety. The assay was performed on serum samples collected on Day +21 and Day +46.

### Example 4

### Efficacy of and Safety Profile of Immunization with SerpinB13

Examine the efficacy of immunization with serpin B13 in preventing type 1 diabetes. An important goal of the present research will be to develop a protocol with better therapeutic efficacy in T1D compared with the serpin peptide vaccine that has been used thus far. Several alternative approaches will be used to provoke active immunization with serpin B13 peptide, either alone, or in combination with anti-serpin B13 mAb and their effects on diabetes development in NOD mice will be examined. Since it is likely that the serpin peptide vaccine is responsible for the intra-pancreatic expansion of T cells with regulatory properties in NOD animals, while anti-serpin antibodies enhance the cleavage of key surface molecules that are expressed in T and B cells (Baldzizhar et al., “Anti-Serpin Antibody-Mediated Regulation of Proteases in Autoimmune Diabetes,” J. Biol. Chem. 288:1612-1619 (2013), which is hereby incorporated by reference in its entirety) this combination may be particularity effective in subduing autoimmune inflammation in the islets. Another alternative will be to combine the serpin-vaccine peptide with anti-CD3 mAb or adjuvant other than alum, or immunize NOD mice with the full-length serpin B13. It will be equally important to examine other methods of antigen delivery for their efficiency to break tolerance to serpins. For example, the anti-diabetic impact of immunization of NOD mice will be examined with replication-defective adenovirus type 5 (rAdS) vectors that express serpin B13. Conventional Ad vectors that express intact serpin B13 (either mouse or a non-murine serpin B13 [e.g., xenoantigen]) or protein subdomains (e.g., AA 265-285 B cell epitope alone (Czyzyk et al., “Enhanced Anti-Serpin Antibody Activity Inhibits Autoimmune Inflammation in Type 1 Diabetes,” J. Immunol. 188:6319-6327 (2012), which is hereby incorporated by reference in its entirety) with or without a T helper epitope such as PADRE) will be constructed, as well as vectors that encode serpin B13 and display it on the surface of the adenovirus capsid. The latter approach is expected to result in more efficient elicitation of anti-serpin B13 antibodies, due to the repetitive, arrayed nature of protein/peptide display on the adenovirus capsid (Bouer et al., “Protective Immunity Against a Lethal Respiratory Yersinia pestis challenge Induced by V Antigen or the Fl Capsular Antigen Incorporated Into Adenovirus Capsid,” Human Gene Ther. 21:891-901 (2010), which is hereby incorporated by reference in its entirety). In an alternative approach, to avoid the non-specific inflammation that adenovirus frequently elicits, nanoparticle scaffolds will be used (Arany et al., “Nanoparticle-Mediated Gene Silencing Confers Radioprotection to Salivary Glands In Vivo,” Mol. Ther. 21:1182-1194 (2013), which is hereby incorporated by reference in its entirety) to which serpin B13 (or irrelevant protein as a control) will be attached, and their effects on T1D in NOD mice will be studied. Moreover, serpin B13 density on nanoparticle surfaces can be tuned to study how multifunctionality affects immune response.

Examine the safety profile of immunization with serpin. Because serpin B13 is not only expressed in the pancreas, but also in other organs (e.g., the skin) it will be critical to determine whether enhancing anti-serpin B13 immunological responses poses a risk of side effects including systemic autoimmunity. Although thus far no serious detrimental changes have been observed following treatment with anti-serpin B13 mAb or serpin peptide (e.g., there were no signs of seizure, weight loss, or skin lesions), a much more detailed search for potential side effects is warranted once an optimal intervention protocol is developed. In addition to clinical evaluation and gross organ examination, histology studies and biochemical tests will be performed on various tissues obtained from serpin vaccinated animals.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. Additionally, the recited order of processing elements or sequences, or the use of numbers, letters, or other designations therefore, is not intended to limit the claimed processes to any order except as may be specified in the claims. These alterations, improvements, and modifications are intended to be suggested hereby, and are within the spirit and scope of the invention. Accordingly, the invention is limited only by the following claims and equivalents thereto.

1. An immunogenic composition comprising a pharmaceutically acceptable carrier and a serpinB13 protein or polypeptide.

2. The immunogenic composition according to claim 1 further comprising an adjuvant.

3. The immunogenic composition according to claim 2, wherein the adjuvant comprises aluminum hydroxide, aluminum phosphate, aluminum potassium sulfate, beryllium sulfate, silica, kaolin, carbon, water-in-oil emulsions, oil-in-water emulsions, muramyl dipeptide, bacterial endotoxin, lipid, Quil A, non-infective Bordetella pertussis, or a combination thereof.

4. The immunogenic composition according to claim 1, wherein the serpinB13 protein or polypeptide is conjugated to an immunogenic carrier molecule.

5. The immunogenic composition according to claim 4, wherein the immunogenic carrier molecule comprises bovine serum albumin, chicken egg ovalbumin, keyhole limpet hemocyanin, tetanus toxoid, diphtheria toxoid, thyroglobulin, a pneumococcal capsular polysaccharide, CRM 197, or a meningococcal outer membrane protein.

6. The immunogenic composition according to claim 1, wherein the serpinB13 polypeptide is present in a fusion protein.

7. The immunogenic composition according to claim 6, wherein the serpinB13 polypeptide is a terminal or internal fragment comprising at least about 10 consecutive amino acids.

8. The immunogenic composition according to claim 6, wherein the fusion protein also comprises an adjuvant polypeptide.

9. The immunogenic composition according to claim 8, wherein the adjuvant polypeptide is flagellin, human papillomavirus (HPV) L1 or L2 protein or polypeptide, herpes simplex glycoprotein D (gD), complement C4 binding protein, toll-like receptor-4 (TLR4) ligand, or IL-1β.

10. The immunogenic composition according to claim 1, wherein the pharmaceutically acceptable carrier comprises an aqueous solution, an oil, a glycol, or a mixture thereof.

11. The immunogenic composition according to claim 1, wherein the serpinB13 protein or polypeptide is a human serpinB13 protein or polypeptide.

12. An immunogenic polypeptide comprising a serpinB13 protein or polypeptide coupled to an immunogenic agent.

13. The immunogenic polypeptide according to claim 12, wherein the immunogenic agent is bovine serum albumin, chicken egg ovalbumin, keyhole limpet hemocyanin, tetanus toxoid, diphtheria toxoid, thyroglobulin, a pneumococcal capsular polysaccharide, CRM 197, or a meningococcal outer membrane protein.

14.-15. (canceled)

16. The immunogenic polypeptide according to claim 12, wherein the immunogenic polypeptide is a fusion protein.

17. The immunogenic polypeptide according to claim 16, wherein the fusion protein also comprises an adjuvant polypeptide.

18.-20. (canceled)

21. The immunogenic polypeptide according to claim 12, wherein the serpinB13 protein is present.

22. The immunogenic polypeptide according to claim 12, wherein the serpinB13 polypeptide is present, and the serpinB13 polypeptide is a terminal or internal fragment comprising at least about 10 consecutive amino acids.

23. The immunogenic polypeptide according to claim 12, wherein the serpinB13 protein or polypeptide is a human serpinB13 protein or polypeptide.

24. A pharmaceutical composition comprising the immunogenic polypeptide according to claim 12.

25. The pharmaceutical composition according to claim 24, further comprising a pharmaceutically acceptable carrier, excipients, diluent, or adjuvant.

26.-27. (canceled)

28. A method of inhibiting or delaying onset, or reducing the severity of, type 1 diabetes, the method comprising:
administering to an individual having a risk of developing type 1 diabetes an effective amount of serpinB13 protein or a polypeptide fragment thereof, an immunogenic composition according to claim 1, or an immunogenic polypeptide comprising a serpinB13 protein or polypeptide coupled to an immunogenic agent wherein said administering is effective to induce an anti-serpinB13 antibody response that inhibits or delays onset of type 1 diabetes, or reduces the severity of type 1 diabetes in the individual.

29. The method according to claim 28, further comprising assessing the individual's risk of developing type 1 diabetes.

30.-43. (canceled)

44. A method of treating an individual for an immune condition comprising:
administering to an individual having an immune condition an effective amount of serpinB13 protein or a polypeptide fragment thereof, or an immunogenic composition according to claim 1, or immunogenic polypeptide comprising a serpinB13 protein or polypeptide coupled to an immunogenic agent wherein said administering is effective to treat the immune condition or control symptoms thereof, and wherein the immune condition is selected from the group consisting of psoriasis, hair loss, and ulcers.

45.-49. (canceled)
